(19) United States
(12) Patent Application Publication
(10) Pub. No.: US 2014/0193466 A1
(43) Pub. Date: Jul. 10, 2014

(54) SILK COMPOSITIONS AND METHODS OF USING SAME

(71) Applicant: CORNELL UNIVERSITY, Ithaca, NY (US)

(72) Inventors: Brian D Lawrence, Minneapolis, MN (US); Alejandro Navas, Colonia Obrera (MX); Mark I. Rosenblatt, New York, NY (US)

(73) Assignee: CORNELL UNIVERSITY, Ithaca, NY (US)

(21) Appl. No.: 14/124,836

(22) PCT Filed: Jun. 7, 2012
(86) PCT No.: PCT/US12/41288

§ 371 Date: Mar. 18, 2014

Related U.S. Application Data
(60) Provisional application No. 61/494,293, filed on Jun. 7, 2011.
(60) Provisional application No. 61/495,167, filed on Jun. 9, 2011.

(51) Int. Cl. A61K 38/17; A61K 45/06; A61K 9/70

(52) U.S. Cl. CPC A61K 38/1767; A61K 9/70; A61K 45/06
USPC 424/400; 514/1.1; 530/353; 514/20.9; 424/93.6; 424/93.7; 514/20.8; 514/18.3; 514/19.2; 428/174; 428/220; 428/195.1

(57) ABSTRACT

The present invention provides for silk-derived compositions for treating a wide variety of ocular conditions. The composition is produced by processing the silk cocoon into a water-based solution (i.e., a dissolved silk), which is then cast into a film. The film may be transparent to visible light, and curved in shape for easy application to the ocular surface. The silk film may either self-adhere or be sutured to cover the wound. The degradation time of the film may range from 1 minute to 24 hours, or from 2 hours to 20 hours. The present compositions can help regenerate damaged corneal tissue, thus promoting healing.

### CROSS REFERENCE TO RELATED APPLICATION

This application claims priority to U.S. Provisional Application Nos. 61/494,293 (filed on Jun. 7, 2011) and 61/495,167 (filed on Jun. 9, 2011), both of which are incorporated herein by reference in their entirety.

### FIELD OF THE INVENTION

The present invention relates to silk fibroin compositions in a variety of medical uses. In particular, the present invention relates to silk fibroin films for treating ocular conditions.

### BACKGROUND OF THE INVENTION

Each year approximately 2.5 million people in the United States receive traumatic injuries to their eyes. A wounded eye can cause extreme pain, swelling, blurred vision, and even vision loss. Roughly 60,000 patients are diagnosed with trauma-related blindness each year. 40% of all cases of blindness are caused by trauma. Whitcher et al. Corneal blindness: a global perspective. Bulletin of the World Health Organization. 2001; 79:214-221. Worldwide, about 50 million eye injuries occur annually with severe injuries being a major problem especially in developing countries. In addition, each year 11 million Americans experience ocular surface disorders, such as corneal ulcers, erosion, degeneration and perforation. Yiu et al. Ocular surface reconstruction: recent advances and future outlook. Current Opinion in Ophthalmology. 2007; 18(6):509. Gomes et al. Amniotic membrane use in ophthalmology. Current Opinion in Ophthalmology. 2005; 16(4):233. Many of these disorders need to be repaired through therapies or surgical procedures. Moreover, procedures such as retinal vitrectomy, photorefractive surgery and cataract removal account for millions of sustained surgical wounds. Sandoval et al. Refractive surgery survey 2004. Journal of Cataract & Refractive Surgery, 2005; 31(1):221-233. Taneri et al. DIAGNOSTIC AND SURGICAL TECHNIQUES. SURVEY OF OPHTHALMOLOGY. 2004; 49(6).

In most cases, treatment options are limited. The available treatments only protect the eye from further injury, thus allowing the wounds to heal. For years, in the hopes of regenerating damaged ocular tissue, researchers have pursued regenerative technologies, such as stem cells therapies. However, these efforts have been slow to come to fruition. Recently, donor amniotic tissue has shown promise in ocular surface regeneration; but its use is limited to the most severe wounds due to its high cost, difficulty in application, and distribution problems. Gomes et al. Amniotic membrane use in ophthalmology. Current Opinion in Ophthalmology. 2005; 16(4):233. Saw et al. Amniotic membrane transplantation for ocular disease: a prospective evaluation of the first 233 cases from the UK user group. British Medical Journal. 2007. Limb et al., Current prospects for adult stem cell-based therapies in ocular repair and regeneration, Current Eye Research, 2006; 31(5):381-90. Grueterich M, Espana E. ScienceDirect.com—Survey of Ophthalmology—Ex vivo expansion of limbal epithelial stem cells: amniotic membrane serving as a stem cell niche. SURVEY OF OPHTHALMOLOGY. 2003. Jingbo Liu HSYFLLSCT, Update on amniotic membrane transplantation, Expert Review of Ophthalmology, NIH Public Access; 2010 Oct. 1; 5(5):645. Other products for treating ocular surface wounds include eye drops, therapeutic contact lenses, and stem cell transplantation. In severe cases, a cornea transplant may be performed to replace the damaged cornea. George A, Larkin D. Corneal transplantation: the forgotten graft. American Journal of Transplantation. 2004; 4(5):678-685.

Silk proteins offer a potential alternative material to current regenerative approaches due to their biocompatibility, tunable properties and transparency. Vepari et al., Silk as a biomaterial, Progress in Polymer Science, 2007; 32(8-9):991-1007. Altman et al., Silk-based biomaterials Biomaterials, 2003; 24(3):401-16. Lawrence et al., Bioactive silk protein biomaterial systems for optical devices. Biomacromolecules, 2008; 9(4):1214-20. Silk generally is a filamentous product secreted by a silkworm or spider. Silkworm silk fibers are constituted from core fibrous proteins (fibroins), which are held together by glue-like proteins (sericins). Chirila et al. Bombyx mori Silk Fibroin Membranes as Potential Substrata for Epithelial Constructs Used in the Management of Ocular Surface Disorders, Tissue Engineering, Part A, Volume 14. Number 7, 2008, 1203-1211. Silks proteins are characterized by a highly repetitive primary sequence that leads to significant homogeneity in secondary structure, i.e., β-sheets in the case of many silks. These types of proteins usually exhibit important mechanical properties, biocompatibility and biodegradability. Silk proteins provide an important set of material options in the fields of tissue regeneration, biomaterials, tissue engineering and drug delivery. Options for genetic manipulations to tailor sequence further facilitate to exploit these natural proteins for biomedical applications. Altman et al., Silk-based biomaterials, Biomaterials, 2003; 24(3): 401-416; Foo et al., Adv. Drug Deliver. Rev. 2002, 54, 1131-1143; Dinerman et al., J. Control Release, 2002, 82, 277-287; Megeed et al., Adv. Drug Deliver. Rev. 2002, 54, 1075-1091; Petrini et al., J. Mater. Sci-Mater. M. 2001, 12, 849-853; Altman et al., Biomaterials, 2002, 23, 4131-4141; Panilaitis et al., Biomaterials, 2003, 24, 3079-3085.

Silk has been used in biomedical applications for centuries primarily for the ligation of wounds. Recently, in multiple areas of the body, such as bone, neural, cartilage, ligament and tendon, silk has been shown to regenerate damaged tissue. Meinel et al., Silk implants for the healing of critical size bone defects, Bone 2005; 37(5):688-698. Kim et al., Dissolvable films of silk fibroin for ultrathin conformal bio-integrated electronics. Nature Materials, 2010, 9: 511-517. Wang et al., Cartilage tissue engineering with silk scaffolds and human articular chondrocytes, Biomaterials, 2006; 27(25):4434-4442. Harkin et al. Silk fibroin in ocular tissue reconstruction. Biomaterials, 32 (2011) 2445-2458. There has also been initial evidence for using silk films in corneal tissue engineering and in ocular surface repair. Silk films have been found to support corneal cell growth, and to develop stratified epithelial cell sheets equivalent to amniotic membrane substrates. Lawrence et al., Silk film biomaterials for cornea tissue engineering, Biomaterials, 2009; 30(7):1299-308. Harkin et al., Silk fibroin in ocular tissue reconstruction, Biomaterials, 2011. Chirila et al., Bombyx mori silk fibroin membranes as potential substrata for epithelial constructs used in the management of ocular surface disorders, Tissue Engineering Part A, 2008; 14(7):1203-11. However, strategies for using silk proteins within the eye have yet to be fully explored within a clinical context.

### SUMMARY

The present invention provides for a degradable composition comprising fibroin, wherein fibroin comprises β-sheet conformation ranging from about 0% to about 30%, or from about 5% to about 15%; and wherein, upon contacting with a body fluid, less than about 20% (w/w) of the composition degrades after about 1 minute, and greater than about 80% (w/w) of the composition degrades after about 24 hours. The present invention also provides for a degradable composition comprising fibroin, wherein the composition comprises about 1% (w/w) to about 12% (w/w) water, or about 5% (w/w) to about 10% (w/w) water; and wherein, upon contacting with a body fluid, less than about 20% (w/w) of the composition degrades after about 1 minute, and greater than about 80% (w/w) of the composition degrades after about 24 hours. The present invention further provides for a degradable composition comprising fibroin, wherein the composition comprises about 1% (w/w) to about 12% (w/w) water; wherein fibroin comprises β-sheet conformation ranging from about 0% to about 30%; and wherein, upon contacting with a body fluid, less than about 20% (w/w) of the composition degrades after about 1 minute, and greater than about 80% (w/w) of the composition degrades after about 24 hours.

Fibroin may comprise α-helical conformation ranging from about 1% to about 80%. In one embodiment, upon contacting with a body fluid, less than about 20% (w/w) of the composition degrades after about 2 hours, and greater than about 80% (w/w) of the composition degrades after about 20 hours. In another embodiment, upon contacting with a body fluid, less than about 10% (w/w) of the composition degrades after about 1 minute, and greater than about 90% (w/w) of the composition degrades after about 24 hours. In still another embodiment, upon contacting with a body fluid, greater than about 80% (w/w) of the composition degrades after about 10 hours. In some embodiments, upon contacting with a body fluid, greater than about 90% (w/w) of the composition degrades after about 10 hours.

Fibroin may be obtained from silkworm (e.g., Bombyx mori) silk, spider (e.g., Nephila clavipes) silk or genetically engineered silk. In the composition, fibroin may be in an amount ranging from about 80% to 100%.

The composition can be transparent or opaque. The composition can take various forms, such as a film, a fiber, a foam, a hydrogel, a matrix, a mesh, a three-dimensional scaffold, a microparticle, a nanoparticle or a mat.

When the composition is a film, the film can have a curved or flat surface. The film's thickness can range from about 1 μm to about 500 μm, from about 10 μm to about 200 μm, or from about 50 μm to about 100 μm. The film may have a tensile strength ranging from about 1 to about 200 MPa, and a tensile modulus ranging from about 0.1 to about 5 GPa. The film may be surface-patterned or smooth.

The composition may further comprise a pharmacologically and/or biologically active agent, such as proteins, peptides, nucleic acids, carbohydrates, glycoproteins, lipoproteins, RNA/protein composites, cells, nucleic acid analogues, nucleotides, oligonucleotides, peptide nucleic acids, aptamers, viruses, small molecules, or combinations thereof. The cell may be epithelial cells, stem cells, smooth muscle cells, skeletal muscle cells, cardiac muscle cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid cells, parathyroid cells, adrenal cells, hypothalamic cells, pituitary cells, ovarian cells, testicular cells, salivary gland cells, adipocytes, precursor cells and mixture thereof. The pharmacologically or biologically active agent may be mixed with the composition, or may be coated on the composition.

The present invention also provides for a method for treating an ocular condition in a subject comprising applying a degradable composition to an eye of the subject, wherein the composition comprises fibroin; wherein fibroin comprises β-sheet conformation ranging from about 0% to about 30%; and wherein, upon contacting with the eye, less than about 20% (w/w) of the composition degrades after about 1 minute, and greater than about 80% (w/w) of the composition degrades after about 24 hours. In one embodiment, upon contacting with the eye, greater than about 80% (w/w) of the composition degrades after about 10 hours.

The ocular conditions that can be treated by the present methods include an ocular surface disorder, corneal ulcer, corneal erosion, corneal abrasion, corneal degeneration, corneal perforation, corneal scarring, an epithelial defect, keratoconjunctivitis, idiopathic uveitis, corneal transplantation, dry eye syndrome, age-related macular degeneration (AMD, wet or dry), diabetic eye conditions, blepharitis, glaucoma, ocular hypertension, post-operative eye pain and inflammation, posterior segment neovascularization (PSNV), proliferative vitreoretinopathy (PVR), cytomegalovirus retinitis (CMV), endophthalmitis, choroidal neovascular membranes (CNVM), vascular occlusive diseases, allergic eye disease, tumors, retinitis pigmen-tosa, eye infections, scleritis, ptosis, miosis, eye pain, mydriasis, neuralgia, cicatrizing ocular surface diseases, ocular infections, inflammatory ocular diseases, ocular surface diseases, corneal diseases, retinal diseases, ocular manifestations of systemic diseases, hereditary eye conditions, ocular tumors, increased intraocular pressure, herpetic infections, ptyrigium (scleral tumor), wounds sustained to ocular surface, post-photorefractive keratotomy eye pain and inflammation, thermal or chemical burns to the cornea, scleral wounds, or conjunctival wounds. The ocular condition may be caused by aging, an autoimmune condition, trauma, infection, a degenerative disorder (such as keratoconus), endothelial dystrophies, and/or a surgery.

The composition may be self-adhered or sutured to the eye. The composition may be a film, a gel, a hydrogel, an ocular implant, a punctal plug, a contact lens, particles, microparticles, nanoparticles, a mucoadhesive formulation, an in-situ forming gel or film, an iontophoresis formulation, a tablet, a rod, fiber mat, fiber, or a patch.

### BRIEF DESCRIPTION OF THE DRAWINGS

FIGS. 1A-1D. (A) Silk solution is cast into a curved silicone rubber mold and (B) then mounted onto a spindle and rotated at a controlled rate. (C) Lab prototype containing 4 spin casting spindles. (D) The spin casting area is covered, the spindles are attached to a variable voltage motor, and a compressor is used to provide a controlled pressurized source of convective air-flow through the chamber.

FIGS. 2A-2B. Spin-cast silk film showing (A) the curved cross-section, and (B) transparency from the en face viewpoint over black print lettering.

FIGS. 3A-3D. (A-B) Silk films spin-casted using 4% silk solution and (C-D) those produced using 8% silk solution.

FIGS. 4A-4D. (A) Effects of chamber air pressure during spin-casting on silk film thickness (n=4, error bars=standard deviation, * indicates p<0.05 compared to 20, 40, & 60 psi. * indicates p<0.05 compared to 20 & 60 psi). (B) Effects of RPM speed of spin casting spindle on silk film thickness for center and periphery areas (n=4, error bars=standard deviation, * indicates p<0.05 compared to all other speeds except 500 RPM in each group. * indicates p<0.05 when compared to all other speeds except 187 RPM in each group. * indicates p<0.05 compared to all other speeds except 187 and 297 RPM in each group). Thickness profile images of silk films produced at (C) 297 RPM and (D) 600 RPM demonstrating thickness uniformity differences between two spin rates.

FIGS. 5A-5C. Increasing heat-annealing time and temperature reduces silk film dissolution rate. (A) Silk films were heated at 165° C. for the indicated time periods. The film dissolved in 1 mL water to different extents after 24 hours. The “white” residual film indicated material that did not dissolve by the tested time. (B-C) Quantitative assessment of silk protein dissolution using the BCA assay for various heating temperatures (80° C.-180° C.) and times (5-120 minutes).

FIGS. 6A-6B. (A) Total silk film dissolution after being heated at 180° C. over varied time periods, and (B) respective percent initial water mass loss content over various heating time (n=3).

FIGS. 7A-7B. (A) FTIR transmission spectrum of silk cocoons, water-processed films, and unprocessed films indicating the change in β-sheet and α-helix secondary structure. (B) Silk film secondary structure at 160° C. for 0, 30, 60, 90 and 120 minutes, and at 180° C. for 0.30, 60, 90 and 120 minutes.

FIGS. 8A-8F. Phase contrast images of corneal epithelial cells on silk film (×10). (A) Rabbit corneal epithelial cells (RCEC), (B) human corneal epithelial cells (HCEC), (C) human corneal-limbal epithelial cells (HCLE). Scanning electron microscopic images of corneal epithelial cells cultured on silk film. (D) RCEC, (E) HCEC, and (F) HCLE. Microvilli on the cell surface and wide connection with the adjacent cells were observed and indicated normal healthy cell development.

FIGS. 9A-9C. (A) The silk film is transparent and administered using forceps, (B) applied directly to the mouse cornea, and (C) then readily adheres as it hydrates.

FIGS. 10A-10C. (A) The silk film is administered using forceps, (B) applied directly to the rabbit cornea, and (C) then observed using slit lamp photography.

FIGS. 11A-11B. (A) Murine corneal epithelium healing detected by fluorescein staining presented for silk treated and non-treated groups. Green fluorescence represents damaged corneal epithelium (shown as light grey areas in the figures). (B) Statistical comparison between the silk film treated and untreated corneas (* indicates p<0.05; n=3; error bars).

FIGS. 12A-12D. Reduced silk film dissolution after incubation in water for a 15-minute period was associated with increasing WA (water-anneal) processing time at (FA) 0, (B) 20, (C) 40, and (D) 240-minutes. FIG. 12A: Films appeared to have completely dissolved without WA processing; FIG. 12B: Films partially dissolved after 20 minutes of processing; FIG. 12C: Films appeared insoluble after 40 minutes and longer; FIG. 12E: Protein assay results confirmed qualitative assessment that the extent of silk film dissolution in water reduced with WA processing time (n=3, error bars=SD).

FIGS. 13A-13H. Silk film initial adhesion to the (A) cornea was evaluated over time with OCT imaging from (B-F) 0 to 4-minutes, (G) 10-minutes, and (H) 45-minutes respectively. (B) The silk film was found to form a standing wave morphology upon initial adhesion, (C-E) then swelled as the material hydrated. (F-G) This was followed by a period of material dissolution as the film reduced in thickness. (G) The film edge showed uniform thickness and remained well adhered to the corneal surface after 10-minutes post application. (H) Insoluble portions of the silk film were measured at 45-minutes showing the remaining silk particulates.

FIGS. 14A-14C. (A) Fluorescein staining images of rabbit corneal healing progression for treated and untreated rabbits over a 48-hour period. (B) Wound area size and (C) healing rate were similar between animals treated with unprocessed silk film and untreated animals (n=3, error bars=SD).

FIGS. 15A-15B. Histological examination of rabbit corneas 7 days after epithelial debridement for (A) untreated and (B) treated animals. The presence of the silk film material did not negatively impact the cornea stroma architecture or the re-epithelialization process. Silk film remnants were not found in the tissue sectioning.

FIGS. 16A-16C. (A) Time course images of fluorescein stained epithelial debridement area for untreated and silk film treated rabbit groups. (B) Wound healing profiles demonstrated a significant reduction in wound size for treated animals over a 42-hour period post-procedure. (C) A statistically significant increase in healing rate was demonstrated over the first 20 hours post-procedure for treated animals when compared to untreated controls. (* indicates p<0.05 compared to untreated controls, n=3, error bars=SD).

FIGS. 17A-17C. (A) Time course images of fluorescein stained epithelial debridement area for untreated and silk film treated rabbit groups, where the film was removed at the 48 hours post-surgery time point as indicated by the white box. (B) The wound healing profile demonstrated an increase in average wound size over time for treated animals over a 48-hour period post-procedure when the silk film was removed as shown as an image inset. (C) A statistically significant increase in healing rate was demonstrated for untreated controls at the 48-hour time point as compared to silk film treated animals (* indicates p<0.05 compared to untreated controls, n=3, error bars=SD).

### DETAILED DESCRIPTION OF THE INVENTION

The present invention provides for silk-derived compositions for treating a wide variety of ocular conditions. The composition contains silk proteins such as fibroin, and is produced by processing silk cocoons into a water-based solution (i.e., a dissolved silk), which is then cast into a film. The film may be transparent to visible light, and curved in shape for easy application to the ocular surface. The silk film may either self-adhere or be sutured to cover any portion of the wound or the whole wound, and act as a transparent bandage for the ocular surface.

The present compositions may also take the forms of fibers, mats, scaffolds, hydrogels, etc. The compositions can help regenerate damaged corneal tissue, thus promoting healing. Without being limited to any specific physiological mechanism, it is believed that the present compositions enhance epithelial migration into the wound site; reduce inflammatory response; and provides a protective barrier.

The present compositions may be degradable or bioabsorbable. The present compositions may not be degradable or bioabsorbable. In the embodiments where the compositions are degradable, the degradation properties may vary. A faster rate of degradation may be helpful in avoiding chronic foreign body reactions that promote a fibrotic response. Conversely, a slower rate of degradation may be useful to allow more time for tissue integration. For example, the film can degrade over a day when treating a corneal abrasion, or can degrade over weeks/months when treating a recurrent epithelial defect or corneal ulcer.

As used herein, the terms “degradation time”, “dissolution time” and “residence time” are interchangeable, and refer to the period of time it takes for greater than 95% (w/w) of the composition to be degraded upon contacting with a body fluid in a subject (e.g., a patient). The degradation time of the present compositions may range from about 1 minute to about 24 hours, from about 10 minute to about 20 hours, from about 30 minutes to about 18 hours, from about 1 hour to about 16 hours, from about 1 hour to about 24 hours, from about 2 hours to about 20 hours, from about 3 hours to about 18 hours, from about 4 hours to about 16 hours, from about 5 hours to about 14 hours, from about 6 hours to about 12 hours, from about 8 hours to about 10 hours, from about 10 hours to about 24 hours, about 10 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, from about 1 minute to about 12 months, from about 30 minutes to about 10 months, from about 1 hour to about 6 months, from about 2 hours to about 4 months, from about 3 hours to about 3 months, from about 4 hours to about 1 month, from about 5 hours to about 3 weeks, from about 6 hours to about 2 weeks, from about 7 hours to about 1 week, from about 8 hours to about 5 days, from about 9 hours to about 3 days, or from about 10 hours to about 2 days.

Upon contacting with a body fluid, less than about 20% (w/w) of the composition degrades after about 1 minute, and greater than about 80% (w/w) of the composition degrades after about 24 hours; less than about 20% (w/w) of the composition degrades after about 2 hours, and greater than about 80% (w/w) of the composition degrades after about 20 hours; less than about 10% (w/w) of the composition degrades after about 1 minute, and greater than about 90% (w/w) of the composition degrades after about 24 hour; less than about 20% (w/w) of the composition degrades after about 1 minute, greater than about 80% (w/w) of the composition degrades after about 10 hours; less than about 20% (w/w) of the composition degrades after about 1 minute, greater than about 90% (w/w) of the composition degrades after about 10 hours; less than about 10% (w/w) of the composition degrades after about 1 minute, greater than about 90% (w/w) of the composition degrades after about 10 hours; less than about 20% (w/w) of the composition degrades after about 1 hour, greater than about 80% (w/w) of the composition degrades after about 10 hours; or less than about 10% (w/w) of the composition degrades after about 1 hour, greater than about 90% (w/w) of the composition degrades after about 10 hours.

The degradation time of the present compositions may also be measured in water or an aqueous solution at temperatures ranging from about 20° C. to about 40° C., from about 22° C. to about 37° C., from about 25° C. to about 37° C., about 25° C., or about 37° C. The degradation of the present compositions may be measured by any suitable methods that can determine the protein level, e.g., UV absorbance, Bradford protein assay, Lowry protein assay, Bicinchoninic acid assay (BCA protein assay), Biuret protein assay, Ninhydrin protein assay, Amido black protein assay or any other suitable methods.

The present compositions contain at least one silk protein, including fibroin, fibroin-related protein, or modified fibroin protein. The silk protein in the present compositions may range from about 10% (w/w) to about 100% (w/w), from about 20% (w/w) to about 95% (w/w), from about 30% (w/w) to about 90% (w/w), from about 40% (w/w) to about 85% (w/w), from about 50% (w/w) to about 80% (w/w), from about 60% (w/w) to about 99% (w/w), from about 70% (w/w) to about 99% (w/w), from about 80% (w/w) to about 99% (w/w), from about 80% (w/w) to about 100% (w/w), from about 90% (w/w) to about 99% (w/w), or from about 80% (w/w) to about 90% (w/w). Higher or lower silk protein content may also be possible.

The water content in the present compositions may range from about 0% (w/w) to about 60% (w/w), from about 0.5% (w/w) to about 50% (w/w), from about 1% (w/w) to about 40% (w/w), from about 1% (w/w) to about 30% (w/w), from about 1% (w/w) to about 20% (w/w), from about 1% (w/w) to about 15% (w/w), from about 1% (w/w) to about 12% (w/w), from about 2% (w/w) to about 10% (w/w), from about 3% (w/w) to about 9% (w/w), from about 4% (w/w) to about 8% (w/w), from about 5% (w/w) to about 7% (w/w), from about 6% (w/w) to about 12% (w/w), from about 5% (w/w) to about 10% (w/w), or from about 5% (w/w) to about 15% (w/w). Higher or lower water content may also be possible.

To suit specific needs, the properties of the present compositions can be adjusted, such as geometrical size, thickness, degradation rate and transparency. This allows for the production of a wide range of custom medical devices designed specifically to the required application.

The present compositions can also be used to coat other materials (e.g., hydrogel, collagen, silicon or combinations thereof or any other material) to accelerate corneal regeneration and reduce pain. In one embodiment, the present compositions improve the therapeutic benefit of non-silk lens by helping it adhere to the surface.

The present compositions contain at least one silk protein or peptide, which may be fibroin or related proteins, or fragments or variants thereof. Fibroin can be obtained from a solution containing a dissolved silk. Silk can be a silkworm silk, e.g., from domesticated silkworm Bombyx mori, a spider silk, e.g. from Nephila clavipes. Other sources of silk include, but are not limited to, other strains of Bombycidae including Antheraea pernyi, Antheraea yamamai, Antheraea mylitta, Antheraea assama, and Philosamia cynthia ricini, as well as silk producing members of the families Saturnidae, Thaumetopoeidae. Lucas et al., Adv. Protein Chem. 13: 107-242 (1958). In general, silks can be produced by certain species in the class Insecta, including the order Lepidoptera (butterflies), and by species in the class Arachnida, including the order Araneae (spiders).

The starting material for fibroin may be cocoons, cocoon filaments, raw silk, silk fabrics, silk yarn, degummed silk, any other partially cleaned silk, etc. This may also include short fragments of raw or sericin-depleted silk.

Silks may also be from a recombinant source, such as silks from genetically engineered cells (e.g., bacteria, yeast, insect or mammalian cells), silks from transgenic plants and animals, silks from cultured cells, silks from cloned full or partial sequences of native silk genes, and silks from synthetic genes encoding silk or silk-like sequences. See, for example, WO 97/08315 and U.S. Pat. No. 5,245,012.

In certain embodiments, the silk used for generation of the present compositions is substantially depleted of its sericin content (i.e., less than about 4% (w/w) residual sericin in the final extracted silk). Alternatively, higher concentrations of residual sericin may be left on the silk following extraction or the extraction step may be omitted. In aspects of this embodiment, the sericin-depleted silk fibroin has, e.g., less than about 1% (w/w), less than about 2% (w/w), less than about 3% (w/w), less than about 4% (w/w), less than about 5% (w/w), less than about 10% (w/w), less than about 15% (w/w), about 1% (w/w) to about 2% (w/w), about 1% (w/w) to about 3% (w/w), or about 1% (w/w) to about 4% (w/w) residual sericin.

Reagents that may be used to remove sericin from silk include, but are not limited to, urea solutions, hot water, enzyme solutions (e.g., papain, etc.). Mechanical methods may also be used to remove sericin from silk fibroin. They include, but are not limited to, ultrasound, abrasive scrubbing and fluid flow.

For example, to remove sericin, B. mori cocoons are boiled in an aqueous solution, for example, for about 10 minutes to about 5 hours, for about 15 minutes to about 3 hours, for about 20 minutes to about 1 hour, or for about 30 minutes. Shorter or longer boiling time periods are also possible. The aqueous solution can be any suitable solution facilitating the removal of sericin, such as Na₂CO₃ in the concentration of about 0.02M. The cocoons are rinsed, for example, with water to extract the sericin proteins.

After sericin is removed, the resulting silk can then be solubilized using a dissolution agent (e.g., a chaotropic agent) to produce a dissolved silk containing fibroin. The dissolution agent may be an aqueous salt solution. Salts useful for this purpose include, but are not limited to, lithium bromide, lithium thiocyanate, calcium nitrate, calcium chloride, cupri-ethylenediamine, sodium thiocyanate, lithium thiocyanate, magnesium nitrate or other magnesium salts, zinc chloride, sodium thiocyanate, other lithium and calcium halides, other ionic species, urea or other chemicals capable of solubilizing silk. For example, the extracted silk is dissolved in about 9 M-about 12 M LiBr solution. The dissolution agent can be in any suitable solvent, including, but not limited to, aqueous solutions, alcohol solutions, 1,1,1,3,3,3-hexafluoro-2-propanol, hexafluoroacetone, and 1-butyl-3-methylimidazolium. These solvents may also be modified through adjustment of pH by addition of acidic of basic compounds.

When the dissolution agent contains a salt, the salt can subsequently be removed by, for example, dialysis. In one embodiment, the silk solution is dialyzed in water for about 2 hours to about 72 hours, or about 6 hours to about 48 hours. For example, a dialysis cassette with a molecular weight cutoff of 3500 Da may be used. Shorter or longer dialysis time periods are also possible. The dialysis membrane can be, for example, cellulose membranes or any other semi-permeable membrane. Any suitable dialysis system may be used. The apparatus used for dialysis can be cassettes, tubing, or any other system.

Alternatively, the dissolution agent can be organic solvents. Such methods have been described in, for example, Li et al., J. Appl. Poly Sci. 2001, 79, 2192-2199; Min, et al. Sen'l Gakkaishi 1997, 54, 85-92; Nazarov et al., Biomacromolecules 2004 May-June; 5(3):718-26. U.S. Pat. No. 8,178,656. The dissolution agent may alternatively be an acid solution (e.g., formic acid, hydrochloric acid, etc.).

During the dissolution process, various parameters may be modified, including, but not limited to, solvent type, silk concentration, temperature, pressure, and addition of mechanical disruptive forces. Mechanical mixing methods employed may also vary, including, for example, agitation, mixing, and sonication.

If necessary, the silk solution may be concentrated by dialyzing against a hygroscopic polymer, for example, polyethylene glycol (PEG), polyethylene oxide, or amylose. The PEG may be of a molecular weight of 8,000-10,000 g/mol and has a concentration of 25-50%. The dialysis may be about 2 hours to about 12 hours. See, for example, PCT application PCT/US/04/11199. It is also possible to change the buffer phase in the dialysis system, altering water purity or adding hygroscopic polymers to simultaneously remove ions and water from the initial silk solution.

Insoluble debris may be removed from the silk solution at any stage by centrifugation or filtration.

The resultant dissolved silk may have a silk protein (e.g., fibroin) concentration ranging from about 1% (w/v) to about 50% (w/v). It may be possible to expand this range to include higher or lower fractions of dissolved silk. The dissolved silk fibroin may have a concentration ranging from about 1% (w/v) to about 50% (w/v), from about 5% (w/v) to about 50% (w/v), from about 1% (w/v) to about 30% (w/v), from about 1% (w/v) to about 5% (w/v), from about 1% (w/v) to about 10% (w/v), from about 1% (w/v) to about 15% (w/v), from about 1% (w/v) to about 20% (w/v), from about 1% (w/v) to about 25% (w/v), from about 1% (w/v) to about 30% (w/v), from about 5% (w/v) to about 10% (w/v), from about 5% (w/v) to about 15% (w/v), from about 5% (w/v) to about 20% (w/v), from about 5% (w/v) to about 25% (w/v), from about 5% (w/v) to about 30% (w/v), from about 10% (w/v) to about 15% (w/v), from about 10% (w/v) to about 20% (w/v), from about 10% (w/v) to about 25% (w/v), from about 10% (w/v) to about 30% (w/v), about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), about 12% (w/v), about 15% (w/v), about 18% (w/v), about 20% (w/v), about 21% (w/v), about 25% (w/v), about 30% (w/v), at least about 1% (w/v), at least about 2% (w/v), at least about 3% (w/v), at least about 4% (w/v), at least about 5% (w/v), at least about 6% (w/v), at least about 7% (w/v), at least about 8% (w/v), at least about 9% (w/v), at least about 10% (w/v), at least about 12% (w/v), at least about 15% (w/v), at least about 18% (w/v), at least about 20% (w/v), at least about 25% (w/v), or at least about 30% (w/v).

As used herein, the terms “a dissolved silk” and “a silk solution” are interchangeable.

The dissolved silk can then be fabricated into a variety of different forms. A film can be produced by drying aqueous solutions of fibroin on a supporting surface, e.g., a hydrophobic surface. The rate and temperature at which the fibroin solution is dried can vary which may affect both the morphology and properties of the films (i.e. surface hydrophilicity, mechanical properties and degradation properties).

The supporting surface can be, for example, part of a mold. The supporting surface can comprise, for example, polydimethylsiloxane (PDMS), silicone, or any other suitable material. The film is then left to dry until some or all the solvent has evaporated to give solid fibroin silk films. The drying step can take place on the bench or in a laminar flow hood. The drying process can be about 6 hours to about 72 hours, about 12 hours to about 48 hours, about 24 hours to about 48 hours, or 24 hours or 48 hours. After drying, the film is mechanically removed from the supporting surface, for example, by using a surgical blade or forceps. See, for example PCT application PCT/US/04/11199.

The film may be prepared using the following method: (a) providing a supporting surface; (b) casting a silk fibroin solution onto the supporting surface; (c) drying the supporting surface until a film forms; and (d) removing the film from the supporting surface.

In certain embodiments, the film is prepared using a spin casting process. The method may comprise the following steps: (a) providing a supporting surface; (b) casting a silk fibroin solution onto the supporting surface; (c) spinning the supporting surface until a film forms; and (d) removing the film from the supporting surface.

The supporting surface may be concave, convex or flat. The supporting surface may be smooth or patterned. In certain embodiments, the supporting surface is a mold having a concave inner surface. The supporting surface may be spun at a fixed rate, for example, ranging from about 100 to about 800 rotations per minute (RPM), from about 200 RPM to about 600 RPM, from about 300 RPM to about 500 RPM, from about 400 RPM to about 600 RPM, or about 500 RPM. The supporting surface may also be spun at varied rates.

Pressurized air may be flown through the supporting surface. The flow rate of the pressurized air may range from about 5 PSI to about 200 PSI, from about 10 PSI to about 150 PSI, from about 20 PSI to about 100 PSI, from about 20 PSI to about 60 PSI, or about 40 PSI.

The film may be flat or curved in shape. The film may be used as a single layer, or more than one layer stacking together, for example, about 2 to about 10 layers, about 3 to about 8 layers, about 4 to about 6 layers, about 2 to about 5 layers, or about 2 to about 3 layers.

The present invention further provides for a method for coating a surface of a substrate with a silk composition comprising: providing a substrate: coating the substrate with a silk solution; and drying the substrate until a film forms. The substrate may be a medical device. Also provided in the present invention is a method of embedding at least one active agent in a silk film, comprising: (a) blending a silk fibroin solution with at least one active agent; (b) casting the silk solution onto a film-supporting surface; and (c) drying the film.

The film may have a thickness ranging from 1 μm to about 500 μm, from 10 μm to about 200 μm, from 10 μm to about 100 μm, from 30 μm to about 50 μm, from 50 μm to about 100 μm, from about 60 μm to about 240 μm, from about 100 μm to about 200 μm, from about 100 μm to about 150 μm, from about 60 μm to about 120 μm, from about 80 μm to about 120 μm, from 10 nm to about 900 nm, from 10 nm to about 800 nm, from 20 nm to about 600 nm, from 100 nm to about 300 nm, or from 10 nm to about 100 nm. Different film thicknesses can be obtained by varying volume of the solution casted, the protein concentration in the solution, the number of layers, etc.

The silk protein(s) and peptide(s) in the present composition may contain the β-sheet, α-helix, random coil, and/or unordered structure.

The silk protein(s) in the present composition may have β-sheet conformation ranging from about 0% to about 90%, from about 1% to about 80%, about 5% to about 70%, about 10% to about 60%, about 20% to about 50%, about 30% to about 40%, about 0% to about 30%, about 1% to about 25%, about 2% to about 20%, about 5% to about 15%, about 8% to about 10%, about 3% to about 12%, about 4% to about 22%, about 10% to about 30%, about 20% to about 40%, about 30% to about 50%, about 40% to about 60%, about 50% to about 70%, about 60% to about 80%, about 10% to about 40%, about 30% to about 60%, about 50% to about 80%, about 40% to about 80%, about 5%, about 10%, about 15%, about 20%, about 25%, about 30%, about 40%, about 50%, about 60%, about 70%, or about 80%.

The silk protein(s) in the present composition may have α-helix conformation ranging from about 0% to about 90%, from about 1% to about 80%, about 5% to about 70%, about 10% to about 60%, about 20% to about 50%, about 30% to about 40%, about 10% to about 30%, about 20% to about 40%, about 30% to about 50%, about 40% to about 60%, about 50% to about 70%, about 60% to about 80%, about 10% to about 40%, about 30% to about 60%, about 50% to about 80%, about 40% to about 80%, about 10%, about 20%, about 30%, about 40%, about 50%, about 60%, about 70%, or about 80%.

The silk protein(s) in the present composition may have random coil conformation ranging about 1% to about 80%, about 5% to about 70%, about 10% to about 60%, about 20% to about 50%, about 30% to about 40%, about 10% to about 30%, about 20% to about 40%, about 30% to about 50%, about 40% to about 60%, about 50% to about 70%, about 60% to about 80%, about 10% to about 40%, about 30% to about 60%, about 50% to about 80%, about 40% to about 80%, about 10%, about 20%, about 30%, about 40%, about 50%, about 60%, about 70%, or about 80%.

Fourier transform infrared spectroscopy (FTIR) may be used to study secondary structure in silk film materials. For example, peaks near 1650-cm⁻¹ and 1550-cm⁻¹ may represent β-sheet and α-helix content respectively. Protein structure may also be measured by x-ray diffraction (XRD), circular dichroism or any other suitable methods.

Besides films, the above-described dissolved silk may also be fabricated into other forms, such as, threads, fibers, foam, meshes, hydrogel, matrixes, three-dimensional scaffolds, tablets, filling material, tablet coating, microparticles, rods, nanoparticles, mats, etc. Methods for generating such are known in the art. See, e.g. U.S. Pat. No. 7,635,755, Altman, et al., Biomaterials 24:401, 2003; PCT Publications WO 2004/000915 and WO 2004/001103; and PCT Application No's PCT/US/04/111199 and PCT/US04/00255, which are herein incorporated by reference.

Fibers may be produced using, for example, wet spinning or electrospinning. Alternatively, a fiber can be pulled directly from a concentrated solution.

Electrospinning can be performed by any means known in the art (see, for example, U.S. Pat. No. 6,110,590). For example, a steel capillary tube with a 1.0 mm internal diameter tip is mounted on an adjustable, electrically insulated stand. The capillary tube is maintained at a high electric potential and mounted in the parallel plate geometry. The capillary tube is connected to a syringe filled with silk solution. A constant volume flow rate is maintained using a syringe pump, set to keep the solution at the tip of the tube without dripping. The electric potential, solution flow rate, and the distance between the capillary tip and the collection screen are adjusted so that a stable jet is obtained. Dry or wet fibers are collected by varying the distance between the capillary tip and the collection screen.

Scaffolds can be produced from aqueous fibroin solutions via a variety of techniques including freeze drying, salt leaching or electrospinning. For example, to produce a scaffold, partially solubilized fibroin fibers may be allowed to dry into a fibrous mat. Scaffolds may also be produced from regenerated aqueous or hexafluoroacetic acid solutions of fibroin. Sponges can be prepared by dehydration of frozen fibroin solutions under vacuum (freeze drying), or by the incorporation of porogens such as salt particles into the fibroin solution. Electrospinning of aqueous fibroin solutions can also be used to create fibrous mats.

Foams may be made from methods known in the art, including, for example, freeze drying and gas foaming (where water may be the solvent, nitrogen or other gas may be the blowing agent). Alternately, the foam is made by contacting the silk fibroin solution with granular salt. The pore size of foams can be controlled, for example by adjusting the concentration of silk fibroin and the particle size of a granular salt (for example, the diameter of the salt particle can range between about 50 μm and about 1000 μm). The salts can be monovalent or divalent, such as NaCl, KCl, CaCl₂, etc. After formation of the foam, the excess salt is then extracted, for example, by immersing in water. The resultant porous foam can then be dried and the foam can be used, for example, as a cell scaffold in biomedical application. See, for example, PCT application PCT/US/04/11199, U.S. Pat. No. 6,423,252, the disclosure of each of which is incorporated herein by reference.

Hydrogels can be prepared by methods known in the art, see for example PCT application PCT/US/04/11199. The sol-gel transition of the concentrated silk fibroin solution can be modified by changes in silk fibroin concentration, temperature, salt concentrations (e.g. CaCl₂, NaCl, and KCl), pH, hydrophilic polymers, and the like. Before the sol-gel transition, the concentrated aqueous silk solution can be placed in a mold or form. The resulting hydrogel can then be cut into any shape, using, for example, a laser. U.S. Patent Publication No. 20110008406.

The compositions of the present invention can be fabricated by any other suitable method, including, for example, fiber spinning, electrospinning, solvent casting, injection molding, thermoforming, extrusion, sheet extrusion, blown film extrusion, compression molding, and the like. U.S. Pat. No. 8,173,163.

The present compositions may also be used as coatings on a substrate. The substrates include, but are not limited to, medical devices, tissue-engineered materials or medical implants (e.g., a dental implant), tissues, regenerated tissues, veterinary devices, or veterinary implants. A silk film may be wrapped or shaped around the substrate. For example, the substrate can be spine cages, stents, dental implants, or hip and knee prostheses.

The present invention also provides for a method of covering a surface of a substrate with a silk composition by providing a film-support substrate; and covering the film-support substrate with a silk fibroin film.

Additionally, the present composition may be a 3-dimensional composite containing two or more silk-based structures to form scaffolds, sponges, or other silk composite, for applications such as drug delivery systems, tissue engineered materials or other biomedical devices. For example, a silk film may be combined with silk fibroin nanospheres or microspheres carrying an active agent to provide sustained release of the active agent. As another example, silk fiber-based composite comprising silk fibers optionally coated with silk fibroin solution or silk gel may be combined with a silk film to provide flexible fibrous materials for use as optical fiber or muscle fibers. Alternatively, silk-based composite may be wrapped or shaped with a silk film around the contour of the silk-based structure.

The different formats of the present compositions may or may not be processed using water, heat, etc. as described above.

The present composition may contain a plurality of pores. The pores may have a mean (or average) diameter in the range of, e.g., about 0.5 μm to about 100 μm, about 0.5 μm to about 80 μm, about 0.5 μm to about 60 μm, about 1 μm to about 50 μm, about 5 μm to about 30 μm, about 0.5 μm to about 5.0 μm, about 0.5 μm to about 10 μm, about 10 μm to about 1,000 μm, about 200 μm to about 800 μm, about 300 μm to about 700 μm, or about 50 μm to about 200 μm.

To introduce pores, fibroin solutions may be casted in the presence of more hydrophilic polymers, such as poly(ethylene oxide) (PEO). PEO with different molecular weights may be used. In one embodiment, a mixture of silk fibroin (e.g., 1%) and polyethylene oxide (PEO, e.g., 0.05%) solutions is prepared to induce pore formation within the silk film matrix. The solution is cast on flat PDMS substrates to produce a film. Post-casting, silk films are water-annealed and then placed into a water bath for 24 hours to leach out the PEO phase. Lawrence et al. Silk film biomaterials for cornea tissue engineering. Biomaterials. 2009 March; 30(7): 1299-1308.

Other suitable materials may also be used to create pores through phase separation. Non-limiting examples of techniques include track etching.

Without further processing (i.e., annealing), films produced from silk fibroin are highly soluble in water, possibly because of dominating random coil protein structures. The structures of the protein can be transformed from random coil to (β-sheet by further processing. This structural transition decreases aqueous solubility and increases degradation time. The processing treatments include, but are not limited to, heating (Hu et al., Macromolecules, 41, 3939-48 (2008)), mechanical stretching (e.g., the film can be drawn or stretched mono-axially or biaxially) (Jin et al., Nature, 424: 1057-61 (2003)), immersion in polar organic solvents (e.g, methanol, propanol) (Canetti et al., Biopolymers—Peptide Sci. 28:1613-24 (1989)), and curing in water or water vapor (Jin et al., Water-Stable Silk Films with Reduced β-Sheet Content, Advanced Functional Materials, 2005; 15(8):1241-1247). Lawrence et al., Effect of Hydration on Silk Film Material Properties, Macromolecular Bioscience, 2010 Apr. 8; 10(4):393-403.

For water processing (i.e., water-annealing), the film may be placed in a vacuum in the presence of water vapor. The film is then dried, e.g., in a laminar flow hood or on the bench. The vacuum can range from about 0 to about 100% vacuum, from about 10% to about 90% vacuum, from about 20% to about 80% vacuum, from about 30% to about 70% vacuum, from about 40% to about 60% vacuum; from about 0 to about 760 Torr, from about 40 Torr to about 700 Torr, from about 70 Torr to about 600 Torr, from about 100 Torr to about 500 Torr, or from about 300 Torr to about 400 Torr. The relative humidity may range from about 0 to about 100%, from about 10% to about 90%, from about 20% to about 80%, from about 30% to about 70%, from about 40% to about 60%, from about 40% to about 85%, from about 30% to about 55%, from about 60% to about 90%, or from about 50% to about 80%. The temperature can range from about 4° C. to about 80° C., from about 10° C. to about 60° C., from about 15° C. to about 50° C., from about 20° C. to about 40° C., about 20° C., about 25° C., or about 30° C. The water processing time can range from about 0 minute to about 48 hours, from about 10 minutes to about 36 hours, from about 30 minutes to about 24 hours, from about 20 minutes to about 40 minutes, from about 1 hour to about 12 hours, from about 2 hours to about 10 hours, from about 3 hours to about 8 hours, or from about 4 hours to about 6 hours.

For heat processing, the temperature and/or duration of the heating can be adjusted. The temperature may range from about 60° C. to about 300° C., from about 80° C. to about 250° C., from about 100° C. to about 200° C., from about 150° C. to about 180° C., from about 160° C. to about 170° C., about 150° C., or about 180° C. Higher or lower temperatures are also possible. The heat processing time may range from about 10 minutes to about 10 hours, from about 30 minutes to about 8 hours, from about 1 hour to about 6 hours, from about 1 hour to about 4 hours, from about 2 hours to about 3 hours, or from about 1 hour to about 2 hours. Longer or shorter processing time periods are also possible. In one embodiment, a dry heat environment (e.g., a dry heat sterilizing oven) is used. In another embodiment, steam heating is used. Additionally, heat-annealing may have the added benefit of sterilizing the material while simultaneously processing the silk composition to increase dissolution time.

The surface of the present composition may be smooth, or may be patterned to guide cell alignment, and facilitate cell adhesion, mobility and proliferation. For example, the film may have an optical pattern, such as a holographic image.

The surface pattern may include any desirable pattern. The surface patterning technique are known in the art, including, for example, ink jet printing of patterns, dip pen nanolithography patterns, microcontact printing or soft lithographic techniques. See Wilran et al., 98 P.N.A.S. 13660-64 (2001); Bettinger et al, 19 Adv. Mat. 2847-50 (2007). Also see PCT/US/07/83620; PCT/US2008/082487. Topographic patterning on the surface of silk film combined with silk film's optical transparent clarity may provide high resolution surface features that are not only suitable for bio-optical device such as an optical grating, a lens, a microlens array (WO 08/127,404), but also suitable for tissue engineered construct due to their ability to direct cellular function and matrix deposition such as tissue alignment and proliferation (WO 08/106,485).

When preparing the film, the supporting surface may have ruled and holographic diffraction gratings with desired grooves/mm spacing. Lawrence et al. Silk film biomaterials for cornea tissue engineering. Biomaterials. 2009 March; 30(7): 1299-1308.

For visible light, the refractive index of the present composition may range from about 1 to about 2, from about 1.3 to about 1.7, or about 1.5. Higher or lower refractive indexes are also possible.

The tensile strength of the present composition may range from about 1 MPa to about 500 MPa, about 50 MPa to about 400 MPa, about 1 MPa to about 200 MPa, from about 5 MPa to about 150 MPa, from about 10 MPa to about 100 MPa, from about 20 MPa to about 80 MPa, from about 30 MPa to about 60 MPa, from about 10 MPa to about 50 MPa.

Elongation at break of the present composition may range from about 1% to about 300%, from about 2% to about 200%, from about 5% to about 150%, from about 10% to about 100%, from about 10% to about 60%, from about 10% to about 30%.

The tensile modulus (or Young's modulus) of the present composition may range from about 0.1 GPa to about 5 GPa, about 1 MPa to about 30 MPa, about 10 MPa to about 50 MPa, about 25 MPa to about 75 MPa, about 50 MPa to about 100 MPa, about 100 MPa to about 300 MPa, about 200 MPa to about 400 MPa, about 300 MPa to about 500 MPa, about 100 MPa to about 500 MPa, about 250 MPa to about 750 MPa, about 500 MPa to about 1 GPa, about 1 GPa to about 30 GPa, about 1 GPa to about 10 GPa, about 10 GPa to about 30 GPa, about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 750 MPa, about 1 GPa, about 5 GPa, about 10 GPa, about 15 GPa, about 20 GPa, about 25 GPa, about 30 GPa, at least 1 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 750 MPa, at least 1 GPa, at least 5 GPa, at least 10 GPa, at least 15 GPa, at least 20 GPa, at least 25 GPa, or at least 30 GPa.

The shear modulus of the present composition may range from about 1 MPa to about 30 MPa, about 10 MPa to about 50 MPa, about 25 MPa to about 75 MPa, about 50 MPa to about 100 MPa, about 100 MPa to about 300 MPa, about 200 MPa to about 400 MPa, about 300 MPa to about 500 MPa, about 100 MPa to about 500 MPa, about 250 MPa to about 750 MPa, about 500 MPa to about 1 GPa, about 1 GPa to about 30 GPa, about 10 GPa to about 30 GPa, about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 750 MPa, about 1 GPa, about 5 GPa, about 10 GPa, about 15 GPa, about 20 GPa, about 25 GPa, or about 30 GPa, at least 1 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 M Pa, at least 750 MPa, at least 1 GPa, at least 5 GPa, at least 10 GPa, at least 15 GPa, at least 20 GPa, at least 25 GPa, or at least 30 GPa.

The bulk modulus of the present composition may range from about 5 GPa to about 50 GPa, about 5 GPa to about 100 GPa, about 10 GPa to about 50 GPa, about 10 GPa to about 100 GPa, or about 50 GPa to about 100 GPa, about 5 GPa, about 6 GPa, about 7 GPa, about 8 GPa, about 9 GPa, about 10 GPa, about 15 GPa, about 20 GPa, about 25 GPa, about 30 GPa, about 35 GPa, about 40 GPa, about 45 GPa, about 50 GPa, about 60 GPa, about 70 GPa, about 80 GPa, about 90 GPa, about 100 GPa, at least 5 GPa, at least 6 GPa, at least 7 GPa, at least 8 GPa, at least 9 GPa, at least 10 GPa, at least 15 GPa, at least 20 GPa, at least 25 GPa, at least 30 GPa, at least 35 GPa, at least 40 GPa, at least 45 GPa, at least 50 GPa, at least 60 GPa, at least 70 GPa, at least 80 GPa, at least 90 GPa, or at least 100 GPa.

The present compositions may, or may not, exhibit optical properties such as transparency and translucency. In certain cases, such as treating an ocular condition (e.g., a bandage for the eye, development of a lens or a “humor” for filling the eye), it would be advantageous to have a transparent material. In some embodiments, the present compositions may be opaque.

In some embodiments, the present compositions are optically transparent. The composition transmits, e.g., about 75% of the light, about 80% of the light, about 85% of the light, about 90% of the light, about 95% of the light, or about 100% of the light, at least 75% of the light, at least 80% of the light, at least 85% of the light, at least 90% of the light, at least 95% of the light, about 75% to about 100% of the light, about 80% to about 100% of the light, about 85% to about 100% of the light, about 90% to about 100% of the light, or about 95% to about 100% of the light.

In certain embodiments, the present compositions are optically opaque. In aspects of this embodiment, the composition transmits, e.g., about 5% of the light, about 10% of the light, about 15% of the light, about 20% of the light, about 25% of the light, about 30% of the light, about 35% of the light, about 40% of the light, about 45% of the light, about 50% of the light, about 55% of the light, about 60% of the light, about 65% of the light, or about 70% of the light, at most 5% of the light, at most 10% of the light, at most 15% of the light, at most 20% of the light, at most 25% of the light, at most 30% of the light, at most 35% of the light, at most 40% of the light, at most 45% of the light, at most 50% of the light, at most 55% of the light, at most 60% of the light, at most 65% of the light, at most 70% of the light, at most 75% of the light, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 25% to about 35%, about 25% to about 40%, about 25% to about 45%, about 25% to about 50%, about 25% to about 55%, about 25% to about 60%, about 25% to about 65%, about 25% to about 70%, or about 25% to about 75% of the light.

In another embodiment, the present compositions are optically translucent. In aspects of this embodiment, the composition diffusely transmits, e.g., about 75% of the light, about 80% of the light, about 85% of the light, about 90% of the light, about 95% of the light, about 100% of the light, at least 75% of the light, at least 80% of the light, at least 85% of the light, at least 90% of the light, or at least 95% of the light, about 75% to about 100% of the light, about 80% to about 100% of the light, about 85% to about 100% of the light, about 90% to about 100% of the light, or about 95% to about 100% of the light.

The present composition may exhibit cohesiveness. In one embodiment, the composition may exhibit strong cohesive attraction, on par with water. In another embodiment, the composition exhibits low cohesive attraction. In yet another embodiment, the composition exhibits sufficient cohesive attraction to remain localized to a site of administration. In still another embodiment, the composition exhibits sufficient cohesive attraction to retain its shape. In a further embodiment, the composition exhibits sufficient cohesive attraction to retain its shape and functionality.

The present compositions may further contain at least one pharmaceutically and/or biologically active agent. The pharmaceutically and/or biologically active agent may possess any desirable properties to suit specific needs. For example, the active agent can enhance proliferation and/or differentiation of cells. The present compositions with at least one active agent may facilitate tissue repair, tissue ingrowth, tissue regeneration, tissue/organ replacement, etc. The present compositions may also be used to deliver an active agent. U.S. Pat. No. 8,071,722.

Non-limiting examples of the pharmaceutically and/or biologically active agents include proteins, peptides, nucleic acids (e.g., DNA, RNA, siRNA, shRNA, antisense RNA, plasmids, etc.), carbohydrates, glycoproteins, lipoproteins, modified RNA/protein composites, cells, nucleic acid analogues, nucleotides, oligonucleotides, peptide nucleic acids, aptamers, viruses, small molecules, and combinations thereof.

Other non-limiting examples of the active agents include anti-infectives such as antibiotics, antimicrobial compounds and antiviral agents; chemotherapeutic agents (i.e. anticancer agents); antibodies or fragments or portions thereof; hormones; hormone antagonists; growth factors and fragments and variants thereof; recombinant growth factors; growth factor inhibitor; cytokines; enzymes; toxins; prodrugs; anti-rejection agents; analgesics and analgesic combinations; anti-inflammatory agents; hormones (e.g., steroids); pharmacological materials; vitamins; sedatives; hypnotics; prostaglandins; radiopharmaceuticals; anti-thrombotics; anti-metabolics; growth promoters; anticoagulants; antimitotics; and thrombolytic drugs.

The active agent may also be cell attachment mediators, such as collagen, elastin, fibronectin, vitronectin, laminin, integrins, selectins, cadherins, proteoglycans, or peptides containing known integrin binding domains. The active agent may include “RGD” integrin binding sequence, or variations thereof; ligands; and substances that enhance or exclude particular varieties of cellular or tissue ingrowth. Schaffner et al., Cell Mol. Life Sci., 2003, January; 60(1): 119-32; Hersel et al. Biomaterials, 2003, November; 24(24):4385-415.

The silk protein, e.g., fibroin, of the present invention may be modified to include desired functional groups (e.g., RGD sequences). Fibroin can be functionalized through, e.g., the lysine residue or tyrosine residue. Chimeric molecules in which fibroin sequences are combined with those found in ECM molecules may also be prepared through genetic engineering.

The present compositions can be shaped into articles for tissue engineering and tissue guided regeneration applications, including reconstructive surgery. The scaffolds may also be molded to form external scaffolding for the support of in vitro culturing of cells for the creation of external support organs. The scaffold may function to mimic the extracellular matrices (ECM) of the body. The scaffold serves as both a physical support and an adhesive substrate for isolated cells during in vitro culture and subsequent implantation. As the transplanted cell populations grow and the cells function normally, they begin to secrete their own ECM support.

A number of different cell types or combinations thereof may be employed in the present invention, depending upon the intended function. These cell types include, but are not limited to, epithelial cells, stem cells, endothelial cells, smooth muscle cells, skeletal muscle cells, cardiac muscle cells, urothelial cells, fibroblasts, myoblasts, chondrocytes, chondroblasts, osteoblasts, osteoclasts, keratinocytes, hepatocytes, bile duct cells, pancreatic islet cells, adrenal cells, hypothalamic cells, pituitary cells, ovarian cells, testicular cells, salivary gland cells, adipocytes, stem cells, osteocytes, neuronal cells, lipocytes, immunocytes, pancreatic Islet cells, exocrine cells, cells of intestinal origin, parathyroid cells, thyroid cells, cells of the adrenal-hypothalamic-pituitary axis, kidney tubular cells, kidney basement membrane cells, nerve cells, blood vessel cells, cells forming bone and cartilage, integumentary cells, bone marrow cells, pluripotent cells, induced pluripotent stem cells, adult stem cells or embryonic stem cells, precursor cells or combinations thereof.

For example, smooth muscle cells and endothelial cells may be employed for muscular, tubular constructs, e.g., constructs intended as vascular, esophageal, intestinal, rectal, or ureteral constructs; chondrocytes may be employed in cartilaginous constructs; cardiac muscle cells may be employed in heart constructs; hepatocytes and bile duct cells may be employed in liver constructs; epithelial, endothelial, fibroblast, and nerve cells may be employed in constructs intended to function as replacements or enhancements for any of the wide variety of tissue types that contain these cells. In general, any cells may be employed that are found in the natural tissue to which the construct is intended to correspond. In addition, progenitor cells, such as myoblasts or stem cells, may be employed to produce their corresponding differentiated cell types. In some instances neonatal cells or tumor cells may be used.

When the active agent is at least one cell, cells could be collected from a multitude of hosts including, but not limited to, donors, human tissues, transgenic mammals, established cell culture lines, or before or after molecular genetic engineering. Pieces of tissue can also be used, which may provide a number of different cell types in a single structure. Cells can be obtained from donors (e.g., allogenic) or from recipients (autologous).

When using the present compositions as a platform to support biologically active agent such as cells, it may be desirable to add other materials to promote the growth of the active agent, promote the functionality of the active agent after it is released from the present composition, or increase the active agent's ability to survive or retain its efficacy during the processing period. Exemplary materials known to promote cell growth include, but are not limited to, cell growth media, fetal bovine serum (FBS), non-essential amino acids and antibiotics, and growth and morphogenic factors such as fibroblast growth factor (e.g., FGF 1-9), transforming growth factors (TGFs), vascular endothelial growth factor (VEGF), epidermal growth factor (EGF), platelet derived growth factor (PDGF), insulin-like growth factor (IGF-I and IGF-II), bone morphogenetic growth factors (e.g., BMPs 1-7), bone morphogenetic-like proteins (e.g., GFD-5, GFD-7, and GFD-8), transforming growth factors (e.g., TGF-α, TGF-β1-111), nerve growth factors, and related proteins. Growth factors are known in the art, see, e.g., Rosen & Thies, Cellular & Mol. Basis. Bone Formation & Repair (R.G. Landes Co.).

Additional material to be embedded in the present composition may include liposomes and related systems for delivery of genetic materials; peptides and proteins to active cellular signaling cascades; peptides and proteins to promote mineralization or related events from cells; adhesion peptides and proteins to improve film-tissue interfaces; antimicrobial peptides; other fibrous proteins, such as collagens, elastins, keratins and myosins, etc.

The amount of the active agent will depend on the particular agent being employed and medical condition being treated. Typically, the amount of active agent represents about 0.001% (w/w) to about 70% (w/w), about 0.001% (w/w) to about 50% (w/w), about 0.001% (w/w) to about 20% (w/w) by weight of the material. Upon contacting with a body fluid, the active agent may or may not be released.

The present composition may contain one or more biocompatible polymers (synthetic or natural), non-limiting examples of which include, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), elastin, glycosaminoclycans, polysaccharides, polyallylamine, cellulose, poly(caprolactone-co-D,L-lactide), S-carboxymethyl keratin, poly(vinyl alcohol) (PVA), hyaluronic acid (U.S. Pat. No. 387,413), pectin (U.S. Pat. No. 6,325,810), polycaprolactone (U.S. Pat. No. 6,337,198), polylactic acid or its copolymers (U.S. Pat. No. 6,267,776), polyglycolic acid or its copolymers (U.S. Pat. No. 5,576,881), polyhydroxyalkanoates (U.S. Pat. No. 6,245,537), dextrans (U.S. Pat. No. 5,902,800), polyanhydrides (U.S. Pat. No. 5,270,419), a cyclodextrin component, polyethylene, polystyrene, polymethylmethylcryalte, polyurethanes, and other biocompatible polymers. See, Liang & Hirabayashi, 45 J. Appl. Polymer Sci. 1937-43 (1992); Arai et al., 84 J. Appl. Polymer Sci. 1963-70 (2002); Kitagawa & Yabuki, 80 J. Appl. Polymer Sci. 928-34 (2001); Noishiki et al., 86 J. Appl. Polymer Sci. 3425-29 (2002); Kesenci et al., 12 J. Biomats. Sci. Polymer Ed. 337-51 (2001); Lee et al., 9 J. Biomats. Sci. Polymer Ed. 905-14 (1998); Tsukada et al., 32 J. Polymer Sci. B, 243-48 (1994); Gotoh et al., 38 Polymer 487-90 (1997); Jin et al., 5 Biomacromols. 711-17 (2004).

The present compositions may also contain one or more other pharmaceutically acceptable components, such as diluents, carriers, excipients, stabilizers, buffers, preservatives, tonicity adjusters, salts, antioxidants, osmolality adjusting agents, emulsifying agents, wetting agents, sweetening or flavoring agents, and the like.

The active agent can be introduced at any point(s) throughout the production process for the present composition. For example, an active agent may be added to an aqueous solution of a silk protein. The solution is then processed to form a silk composition (e.g., a film). Alternatively, the active agent may be loaded into or coated onto the composition after it is formed. The coating can be applied through absorption or chemical bonding. The active agent may be present as a liquid, a finely divided solid, or any other appropriate physical form before being embedded into or coated onto the present compositions. When the active agent is at least one cell, the cells could be seeded on the surface of the present composition, or blended into the dissolved silk.

When the active agent is a therapeutic agent, it may be delivered to the patient locally or systemically. The therapeutic agent may be delivered in an immediate release or a controlled release fashion. A controlled release of the active agent from the present composition may occur over time, for example, about 12 hours to 24 hours; about 12 hours to 42 hours; about 12 to 72 hours; about 3 days to about 7 days; about 8 days to about 15 days; about 15 days to about 30 days. In another embodiment, release may occur for example on the order of about 1 day to 15 days. The controlled release time may be selected based on the condition treated. U.S. Patent Publication No. 20100028451.

The present invention further provides methods for treating an ocular condition in a subject by applying the present composition to an eye of the subject. Upon contacting with the eye, the composition has the above-discussed degradation properties.

Ocular conditions subject to treatment according to the method of the present invention include, but are not limited to, epithelial defects, ulcers, herpetic infections, ptyrigium (scleral tumor), idiopathic uveitis, corneal transplantation, dry eye syndrome, age-related macular degeneration (AMD, wet and dry), diabetic eye conditions, blepharitis, glaucoma, ocular hypertension, post-operative eye pain and inflammation, posterior segment neovascularization (PSNV), proliferative vitreoretinopathy (PVR), cytomegalovirus retinitis (CMV), endophthalmitis, choroidal neovascular membranes (CNVM), vascular occlusive diseases, allergic eye disease, tumors, retinitis pigmentosa, eye infections, scleritis, ptosis, miosis, eye pain, mydriasis, neuralgia, aging (e.g. muscle relaxants and other aesthetic products), cicatrizing ocular surface diseases, ocular infections, inflammatory ocular diseases, ocular surface diseases, corneal diseases, retinal diseases, ocular manifestations of systemic diseases, hereditary eye conditions, ocular tumors, increased intraocular pressure, Stevens-Johnson syndrome; cicatricial pemphigoid; injury caused by thermal or chemical burns; injury caused by contact lens wear and toxicity of preservatives; injury caused by chronic use of topical medication; bacterial or viral infections (e.g., trachoma); severe dry eye syndrome; tumors; epithelial defects on the cornea surface; thermal or chemical burns to the cornea; scleral and conjunctival wounds; wounds introduced by physical injury; or postsurgical complications (surgical procedures may include, but are not limited to, vitrectomy, retinal injection, cataract removal, photorefractive keratotomy (PRK), and glaucoma related procedures).

The ocular conditions may be caused by aging, an autoimmune condition, trauma, infection, a degenerative disorder (such as keratoconus), endothelial dystrophies, and/or a surgery.

The present compositions could protect the injury site, reduce pain by covering exposed nerve endings, reduce eyelid friction against the injury site, and seal puncture wounds sustained to the ocular surface. The composition could be used as a treatment for most wounds to the eye due to its ease of application outside of the operating room and biocompatibility. The compositions can adhere to or be sutured to the eye.

The present compositions are useful for ocular biomedical devices and ocular tissue engineering. For example, in corneal tissue engineering, the surface of the composition supports the corneal fibroblast attachment and proliferation. The composition may be used for in vivo cornea tissue repair or in vitro cornea tissue regeneration for subsequent implantation. Additional exemplary applications of the present compositions include, but are not limited to, fabrication of soft contact lenses, intraocular lenses, glaucoma filtration implants, keratoprostheses, scleral buckles, viscoelastic replacement agents, and eye lens replacement. Lawrence et al., Bioactive silk protein biomaterial systems for optical devices, Biomacromolecules, 2008; 9(4):1214-1220.

The composition may be fabricated as a film, a gel, a hydrogel, an ocular implant, a punctal plug, a contact lens, particles, microparticles, nanoparticles, a mucoadhesive formulation, an in-situ forming gel or film, an iontophoresis formulation, a tablet, a rod, a fiber mat, a fiber, or a patch.

In treating ocular conditions, localized treatments include contacting the eye with a composition of the present invention. The compositions can be implanted into the eye tissue or applied directly to the surface of the eye, e.g., topically, injected periocularly, or intravitreally inserted into ocular tissue. Systemic treatment methods include contacting a patient with a composition of the present invention in the vicinity of the eye so that the drug is delivered systemically to the eye for treatment of an ocular condition. Exemplary treatment forms for systemic administration include dermal patches, subcutaneous implants, gels, ointments, etc.

The present compositions may expedite the healing process by promoting tissue regeneration. The present compositions may or may not be bioabsorbable. Applications of the present compositions include the regeneration of tissues such as ocular, nervous, musculoskeletal, cartilaginous, tendenous, hepatic, pancreatic, integumentary, arteriovenous, urinary or any other tissue.

The present composition may be used for a number of medical purposes including, but not limited to, wound dressing materials, organ repair, organ replacement or regeneration, an active agent delivery device (including immediate and controlled release systems), wound closure systems (including vascular wound repair devices), hemostatic dressings, patches and glues, sutures, coatings, composites, wound protection, cell culture substrate, enzyme immobilization, tissue engineering applications (such as, scaffolds for tissue regeneration, ligament prosthetic devices, and products for long-term or bio-degradable implantation into the human body), and tissue space fillers (such as, a dermal filler). U.S. Pat. No. 6,175,053.

In one aspect, the present composition comprising cells can be used in methods of promoting wound healing or wound closure, for example, at an incision site. The methods may comprise applying the present composition at the wound or incision site and allowing the wound or incision to heal while the present composition is degraded and/or absorbed in the body and is replaced with the patient's own viable tissue. The methods may further comprise the step of seeding the present composition with viable cellular material, either from the individual or from a donor, prior to or during application of the present composition. U.S. Patent Publication No. 20110223153. WO 2005/123114. WO 2008/127402. WO2004/0000915. WO2008/106485.

The present compositions may be used in humans, or in animals such as, dogs, cats, horses, monkeys, pigs, cows, or any other mammals. The present compositions may also be used in other subjects, such as mice, rabbits, etc. U.S. Pat. No. 7,842,780.

The present intention may be sterilized using conventional sterilization process such as radiation based sterilization (i.e. gamma-ray), chemical based sterilization (ethylene oxide), autoclaving, or other appropriate procedures. After sterilization the biomaterials may be packaged in an appropriate sterilize moisture resistant package for shipment and use in hospitals and other health care facilities.

### EXAMPLES

The Examples below are illustrative of compositions and methods of the present invention and are not to be construed as limiting.

### Example 1

### Preparing Curved Silk Films

### Silk Solution Preparation

Bombyx mori cocoons were boiled for 30 min in an aqueous solution of 0.02 M Na₂CO₃, and then rinsed thoroughly with water to extract the sericin proteins, using methods previously reported. Jin et al., Adv. Funct. Mater. 2005, 15 (8), 1241. The solution was then dissolved in 9.3 M LiBr solution at room temperature, yielding a 20% (w/v) solution. This solution was dialyzed in water using a dialysis cassette with a molecular cutoff weight of 3500 Da for 48 h.

### Silk Film Preparation

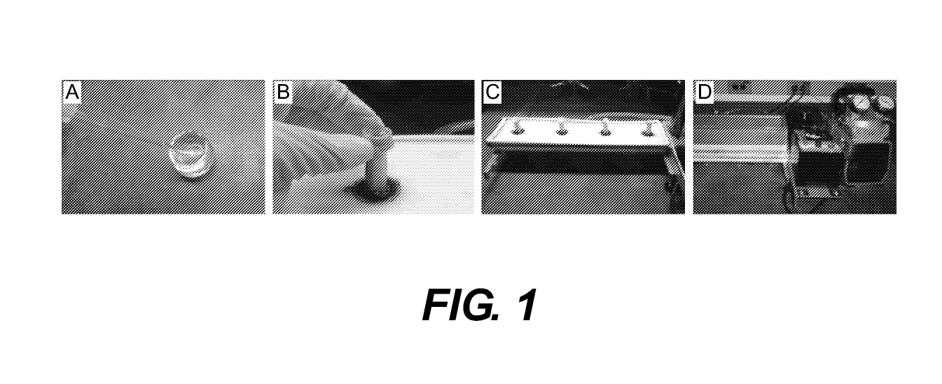

To produce a curved silk film bandage, a spin casting process was developed. A prototype spin casting device was built in which a curved silicone rubber mold (FIG. 1A) could be mounted onto 1 of 4 spindles that were connected to a variable speed motor through a power transfer belt system (FIGS. 1B-1C). To produce the films, silk solution was pipetted into the curved molds and then spun at a fixed rate for a 1.5-hour period until the solution dried. In addition, the curved film drying time was expedited by controlling the spindle environment through controlled compressed air through the system (FIG. 1D).

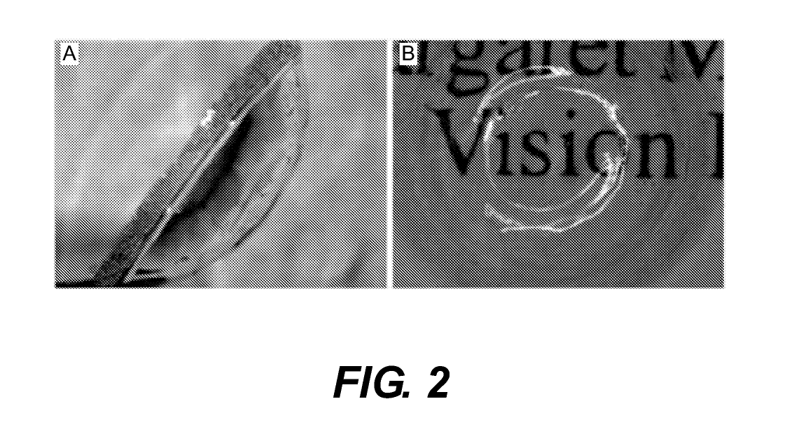

The dried curved film was removed by bending the silicone rubber mold and air-lifting the curved film from the casting surface with forceps. The films that emerged were both curved in shape and highly transparent (FIGS. 2A-2B). The process was found to be highly reproducible and controlled by optimizing the spin cast process parameters (e.g., air flow, RPM, and silk concentration).

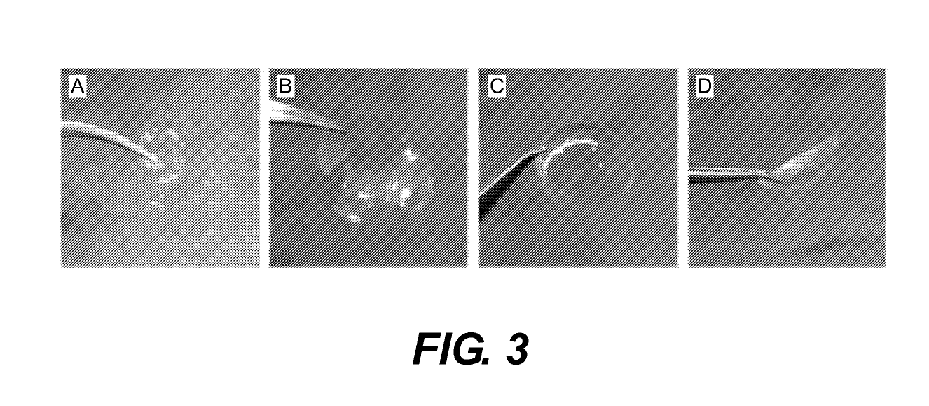

The concentration of the silk solution was found to affect the film produced. Solutions containing 4% silk did not wet the silicone rubber surface very well during the spin cast process (FIGS. 3A-3B). 8% silk solution produced a uniform and well shaped curved silk film bandage (FIGS. 3C-3D).

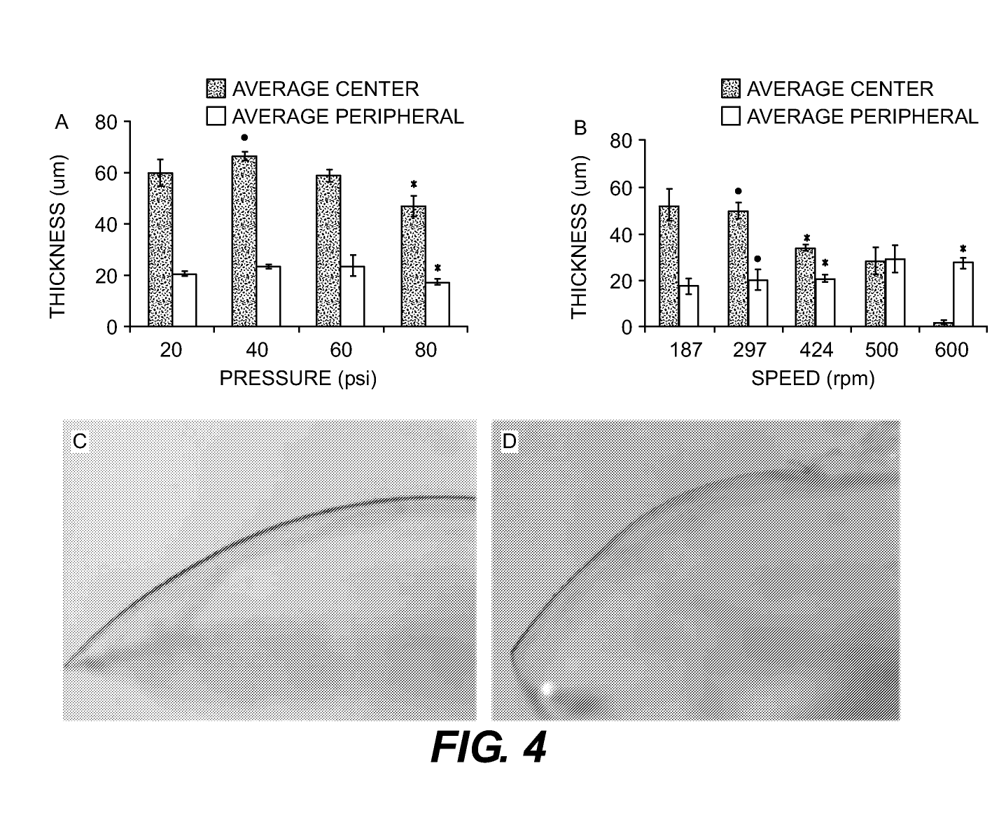

To reduce silk solution drying time, the casting chamber was also vented with pressurized air. It was shown that the introduction of air-flow reduced drying time from 180 minutes down to 90 minutes (50% reduction). The addition of the pressurized air effected silk film bandage thickness (FIG. 4A). The effect of the pressurized air on drying rate was negligible in that as long as vented air was flowing through the system drying time was decreased. The higher flow rates of 60 and 80 PSI produced thinner silk film bandage thickness profiles when compared to 40 PSI. In addition, 20, 40, and 60 PSI showed minimal differences in the center thickness while more significant differences where observed in the periphery regions of the film. 40 PSI was used as a spin casting pressure for other procedures.

The rotation speed (rotations per minute, RPM) of the mold also affected silk film thickness. 187, 297, 424, 500 and 600 RPM rotation settings were tested (FIG. 4B). Lower RPM settings produced films with a thicker center thickness and a thinning periphery thickness (FIG. 4C), while films produced at higher RPM had relatively thick peripheries and thin, or even non-existent, center thickness (FIG. 4D). 500 RPM was used to produce the silk films in other procedures.

### Example 2

### Heat Processing of Film

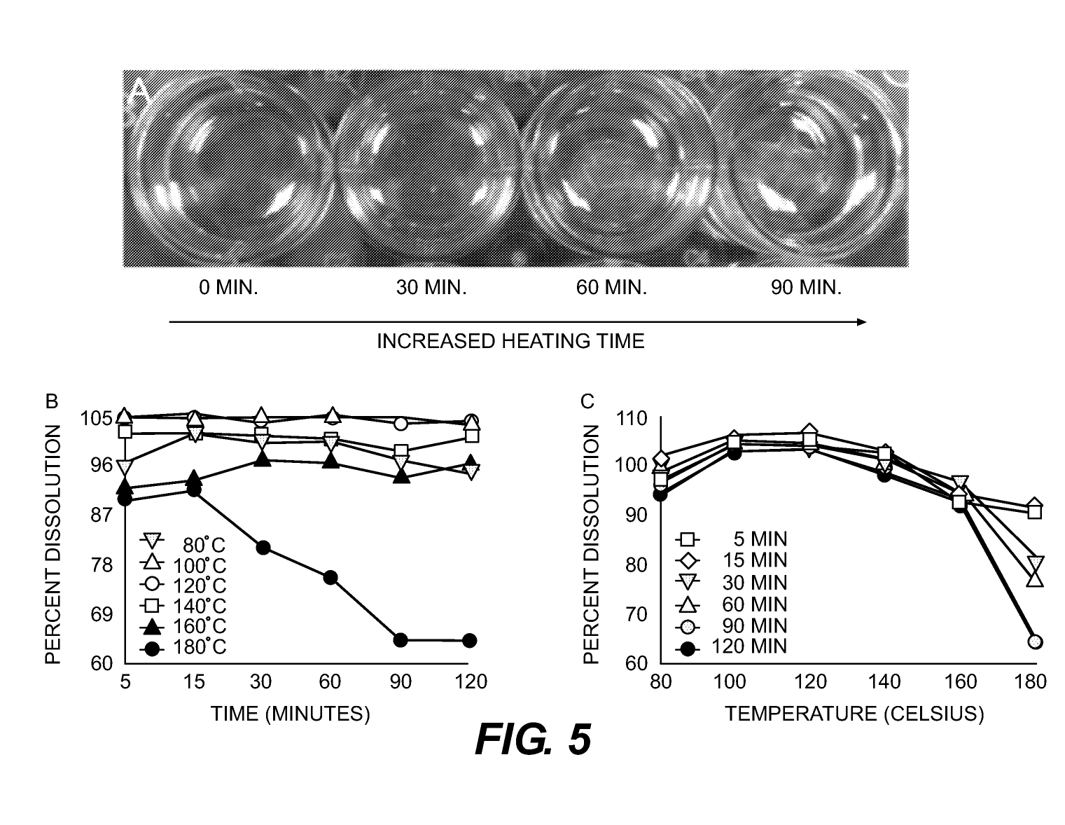

A silk film can be water-annealed to decrease its water solubility. As an alternative to water-annealing, the use of heat-annealing was explored. Heat-annealing is the use of a dry heat environment (i.e. dry heat sterilizing oven) to induce protein secondary structure changes over time, which was shown to increase silk bandage dissolution time qualitatively (FIG. 5A). Additionally, heat-annealing has the added benefit of sterilizing the material while simultaneously processing the silk bandage to increase dissolution time, thus simplifying the manufacturing process by combing two processing steps together. It was found that silk film dissolution time could be readily varied using a window of FDA recommended sterilization temperature (150° C. to 180° C.) and time ranges (1 hour to 2 hours). Silk film dissolution could be quantified using the bicinchoninic acid (BCA) protein content assay. The processed silk film bandages were placed in 1 mL of water, dissolved for 15 minutes, and then sampled for protein content. Assay results indicated that there is an estimated 10% reduction in total protein dissolution between 80° C. and 160° C., with a 25% reduction in dissolution between 160° C. and 180° C. (FIG. 5B). Results for the assay indicated that silk film bandage dissolution could be readily modified based on the length of heat-annealing time with the largest change in dissolution represented at 180° C. (FIG. 5C).

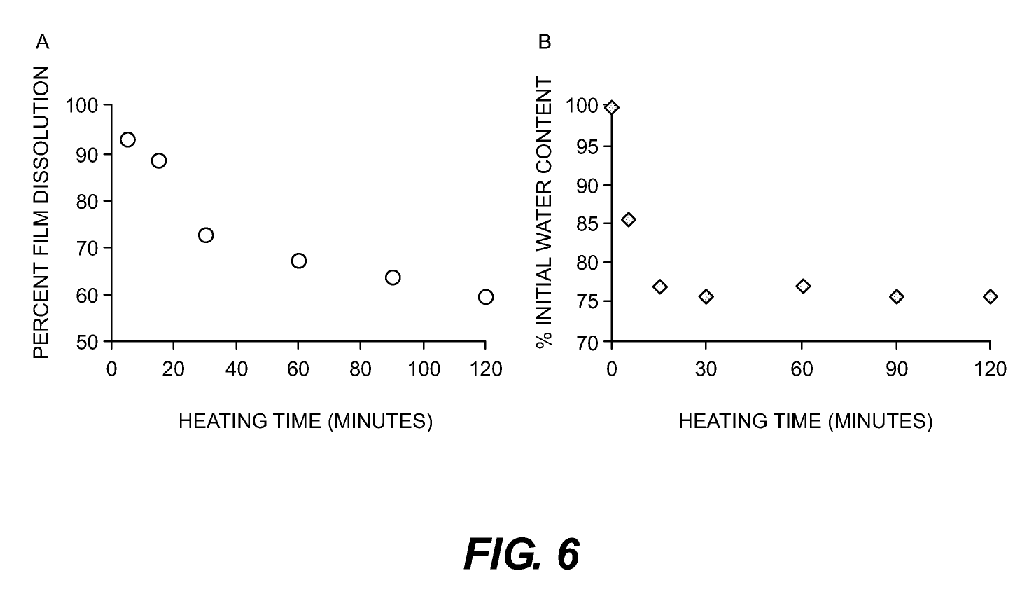

Thermal gravimetric analysis (TGA) was undertaken to study how retained material water content could affect dissolution rate. It was found that percent changes in film dissolution correlated to percent decrease in initial water content with increasing heating time at 180° C. (FIGS. 6A-6B). The results correspond with previous findings that silk film contains bound water molecules that are released at higher heating temperatures. Lawrence et al., Effect of Hydration on Silk Film Material Properties, Macromolecular Bioscience, 2010 Apr. 8; 10(4):393-403. The loss in bound water has been shown to affect protein chain movement and interaction previously, and our current data appears to indicate that loss of material bound water effects overall material solubility. Therefore by controlling total bound water content the data indicates this is also a control point for modifying material dissolution.

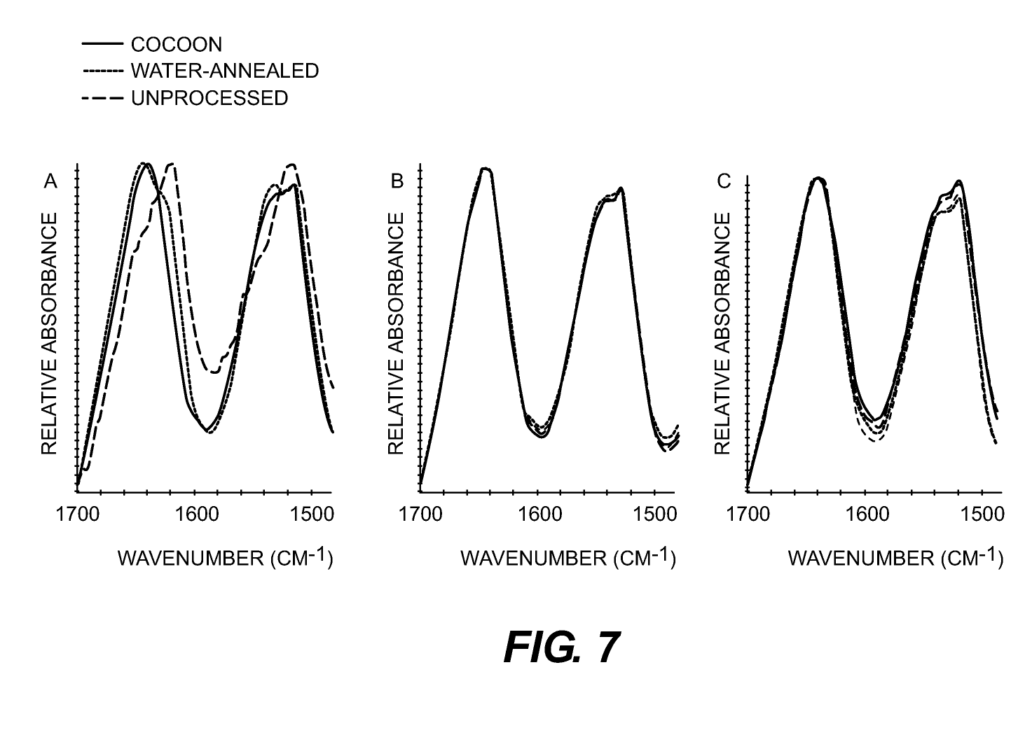

Fourier transform infrared spectroscopy (FTIR) is a useful tool for identifying secondary structure changes in silk film materials. FTIR was undertaken on the silk films heated at various temperatures over various time points. Initial control studies were undertaken that demonstrated a change in protein secondary structure for different material forms of silk worm cocoon, water-annealed silk films, and unprocessed silk films (FIG. 7A). Significant peak changes are evident near 1650-cm⁻¹ and 1550-cm⁻¹ for β-sheet and α-helix content respectively. Peak shifts were not evident for films heated at 160° C. and 180° C. over varying times (FIGS. 7B-7C).

### Example 3

### Silk Film Supports Growth and Proliferation of Corneal Epithelial Cells in Vitro

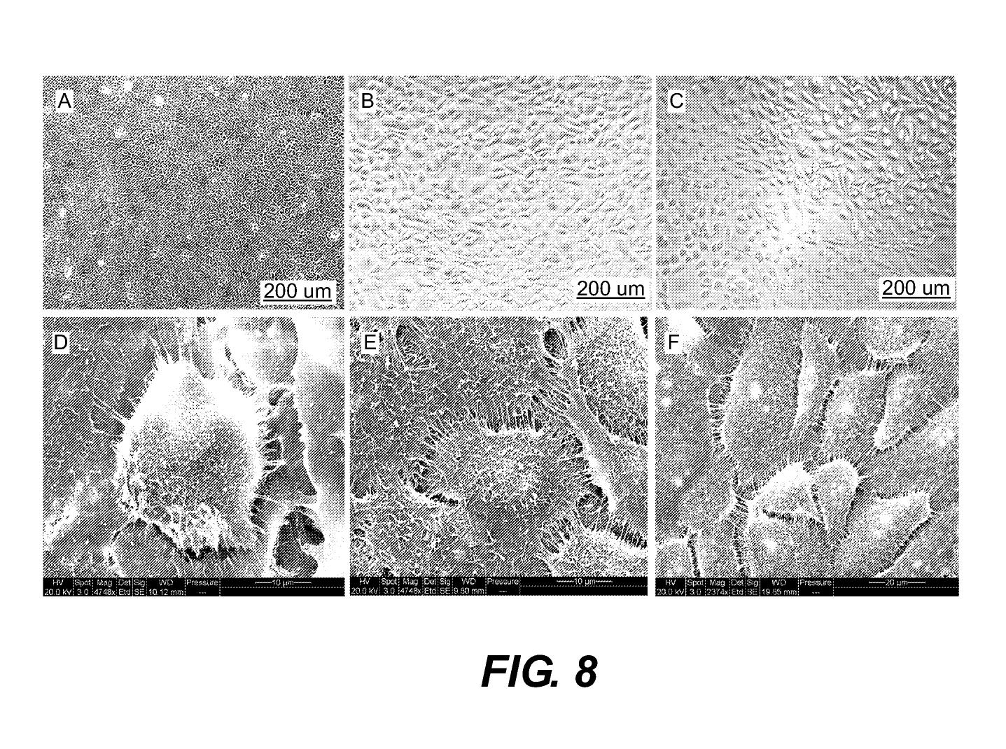

Primary rabbit, primary human, and human cell line corneal epithelial cells were cultured on these films and rapidly generated epithelial cells of normal shape and size (FIGS. 8A-8C). Prominent epithelial cell-to-cell and cell-to-surface contacts were found during scanning electron imaging indicating normal cell structure and healthy culture development (FIGS. 8D-8F). These results indicate that corneal epithelial cells can be successfully cultured on the silk film substrates, and that the material is not cytotoxic to multiple animal cell lines and under a multitude of culture medium conditions.

### Example 4

### Tissue Adhering Properties of Silk Film

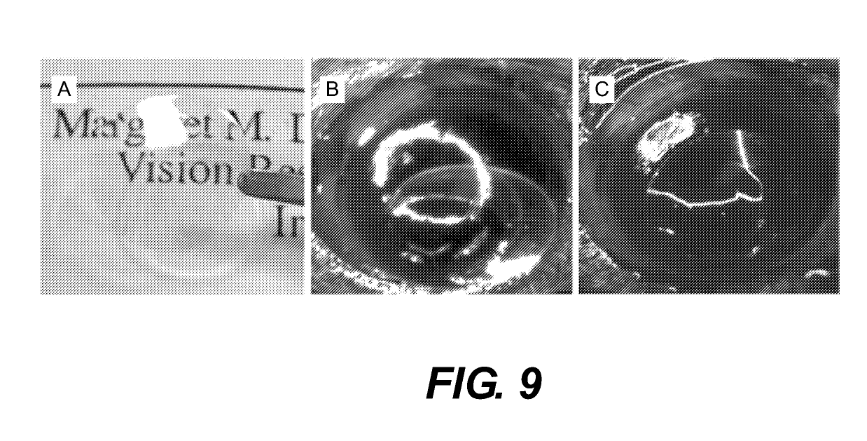

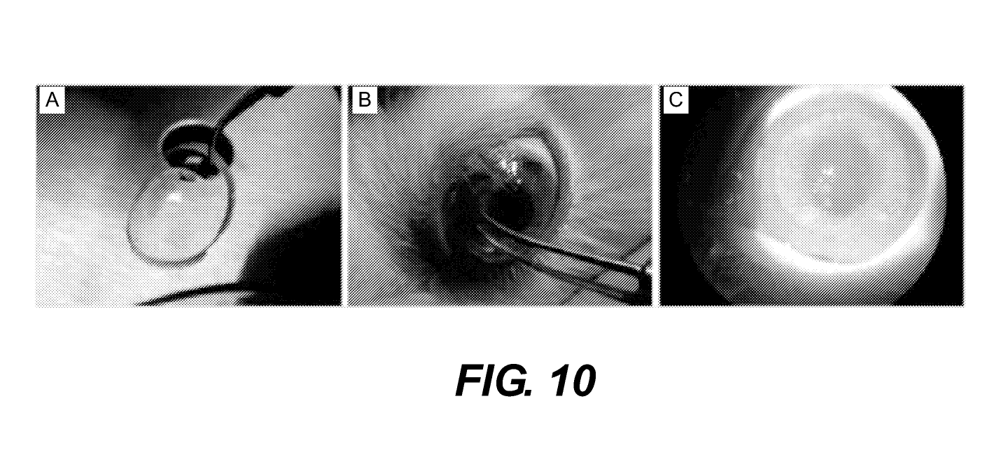

Self-adhesion allows for non-invasive and simple application of the regenerative film because no sutures or adhesives are required. The curved silk film bandage could be readily applied to both the mouse (FIGS. 9A-9C) and rabbit eyes (FIGS. 10A-10B) and then monitored using slit lamp photography (FIG. 10C). When water was absorbed by the film upon placement to the ocular surface the film turned into a gelatinous material due to dissolution of the silk protein back into water. This gelatinous material remained adhered to the cornea surface and provided a protective covering over the wound site.

### Example 5

### Mouse Animal Model

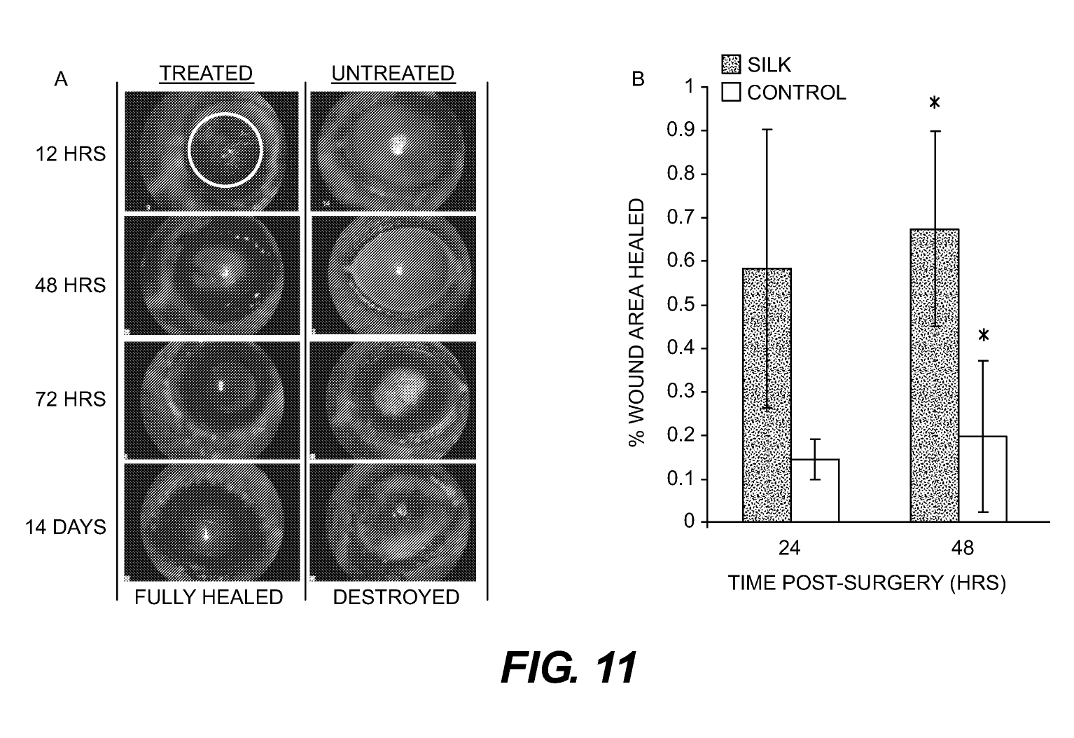

The effectiveness of using the regenerative film for use in repairing an ocular surface wound was investigated in murine animal studies. An ocular surface injury was produced upon the corneas of 6 mice by exposing the corneal epithelium to 30% EtOH and then mechanically removing the epithelial layer with a scalpel. The injury site was then either covered with the unprocessed (i.e., non-annealed) silk film for about 1 day or left uncovered as controls. The extent of corneal injury was assessed in each animal using fluorescein staining which indicates the presence of corneal tissue damage (FIG. 11A). After 48-hours, the silk treated eyes presented a statistically significant increase in healing rate when compared to controls (n=3, p<0.05) (FIG. 11B). After 14-days, the silk treated eyes were found to be completely healed and free of corneal defects while the untreated corneas were severely blinded due to extensive scarring, inflammation, and angiogenesis in the cornea tissue region. These results indicate that the silk film can be used as treatment to expedite epithelial healing rate after an injury to the ocular surface. In addition, the study shows that the film can potentially act to help stop the onset of blindness by reducing the amount of inflammation experienced after an ocular trauma has occurred.

### Example 6

### Rabbit Animal Model

### Materials and Methods

### Production of Silk Solution

Bombyx mori silkworm cocoons (Tajima Shoji Co., Yokohama, Japan) were cut into thirds and then boiled for 40 minutes in 0.02M Na₂CO₃ (Sigma-Aldrich) to extract the glue-like sericin proteins from the structural fibroin proteins as previously described. Lawrence et al., Silk film biomaterials for cornea tissue engineering. Biomaterials. 2009; 30(7): 1299-308. The fibroin extract was then rinsed three times in dH₂O for 20 minutes per wash then dried overnight. The rinsed fibroin extract was then dissolved in 9.3M LiBr solution at room temperature, and placed covered in a 60° C. oven for 4 hours. The solution was dialyzed in water for 48 hours (MWCO 3,500, Pierce, Inc.). The dialyzed silk solution was centrifuged twice at 13,000 g, and the supernatant collected and stored at 4° C. The final concentration of aqueous silk solution was 8 wt./vol. %, as determined by gravimetric analysis.

### Preparation of PDMS Casting Surfaces

Flat polydimethylsiloxane (PDMS) substrates of 0.5 to 1.0 mm thickness were produced by pouring 5 mL of a 1:10 casting catalyst/potting solution (Momentive, Inc., Albany, N.Y.) onto a plastic 90-mm petri dish surface. The cast PDMS solution was then degassed for 2 hours under vacuum, and then cured in an oven at 60° C. overnight. The following day the cured PDMS was removed from the silicon substrate and then punched to form round 14-mm circles. The PDMS substrates were placed cast side up and dust/debris was cleared by using clear tape. The surfaces were further cleaned with 70% ethanol, three dH₂O rinses, and then allowed to air dry in a clean environment.

### Silk Film Casting and Sterilization

Silk films measuring 80 μm in thickness were created by casting 200 μL of 8% silk fibroin solution upon the round PDMS surface. After casting the silk solution, films were covered and allowed to dry for 24 hours to form the patterned silk film surface. Silk film samples were then water-annealed (WA) at different time points by placing the samples in water filled chambers at a 10-psi vacuum and 85% relative humidity. Three sets of samples were evaluated that were annealed for 0, 20, 40 or 240 minutes to produce varying degrees of silk solubility. Longer processing time may be associated with higher β-sheet protein secondary structure and greater hydrophobicity. Jin et al., Water Stable Silk Films with Reduced β-Sheet Content. Advanced Functional Materials, 2005, 15(8):1241-7. Hu et al., Regulation of Silk Material Structure by Temperature-Controlled Water Vapor Annealing, Biomacromolecules, 2011 May 9; 12(5): 1686-96.

### In Vitro Silk Film Dissolution Testing and Optical Coherence Tomography (OCT) Imaging of Initial Silk Film Adhesion In Vivo

Qualitative assessment of silk film dissolution was carried out by placing samples that were water-annealed for 0, 20, 40 and 240 minutes respectively into 4 mL of dH₂O for 15 minutes. Images of silk film dissolution were taken on a stereomicroscope (SteREO Lumar. V12, Carl Zeiss Microscopy, Germany) to assess material solubility. In addition, the Bradford protein content assay was performed to quantitatively assess silk dissolution. Briefly, silk films from each processing time sample group (n=3) were placed into individual QIAshredders (Quiagen, Valencia, Calif.). The centrifugal port of the QIAshredder was covered with parafilm to prevent premature solution flow through, and 500 μL of dH₂O was added to the QIAshredder to begin silk dissolution. The silk films were then mixed for 15 minutes, the parafilm stoppers removed, and then centrifuged. A 1:10 dilution of silk supernatant was prepared, and a 1:5 dilution of Bradford stock reagent (BioRad, Hercules, Calif.) was prepared. The assay was ran by preparing a 1:20 dilution of sample to reagent dilution in a 96-well plate, and absorbance was read at 595-nm.

Initial attachment of unprocessed silk films to the corneal surface was observed using OCT imaging of the silk film attaching to an uninjured corneal surface, and then monitored over time with a Bioptigen SDOIS system (Research Triangle Park, N.C.). Before applying the silk film, the rabbit's eye was treated with the topical anesthetic proparacaine.

### Histology

Excised rabbit corneas were prepared for hemotoxylin and eosin (H&E) staining. Samples were paraffinized, and then sectioned into 7-μm thick slices. The sections were deparaffinized with two changes of Histoclear solution (National Diagnostics, Atlanta, Ga.), and then serial rehydrated in serial ethanol dilutions. Samples were stained in hemotoxylin and differentiated in 1% acid alcohol, and then blued in 0.2% ammonia water. Samples were counterstained in eosin solution, serially dehydrate with Ethanol dilutions, and mounted with DPX mounting medium.

### In Vivo Injury Model and Analysis

Animals were handled according to the ARVO Statement for the Use of Animals in Ophthalmic and Visual Research, under protocols approved by the Institutional Animal Care and Use Committee at the Weill Cornell Medical College. After anesthetizing the rabbit with a ketamine/xylazine solution topical proparacaine anesthetic was applied to the eye, and a speculum was placed to maintain the eyelids open. A trephine 8-mm in diameter was used to demarcate the cornea. Epithelial debridement was performed within the marked area with a #15 surgical blade. A silk film was then applied to the corneal surface and allowed to self-adhere, or no film was applied for untreated controls. A drop of topical moxifloxacin antibiotic was applied to the eye, and rabbits closely monitored for evidence of distress or infection. The wound healing of the rabbits was monitored and examined using a slit lamp microscope. The corneal wounds were then measured using a 1 mg/mL concentration of fluorescein dye (Sigma, Inc.) at 24, 48, and 72-hour time points. Fluorescein staining indicates a de-epithelized surface as denoted by green fluorescence under blue light (shown as light grey areas in the figures). The data was statistically analyzed using the Student t-test.

### Results

### Silk Film Production and Self Adhesion

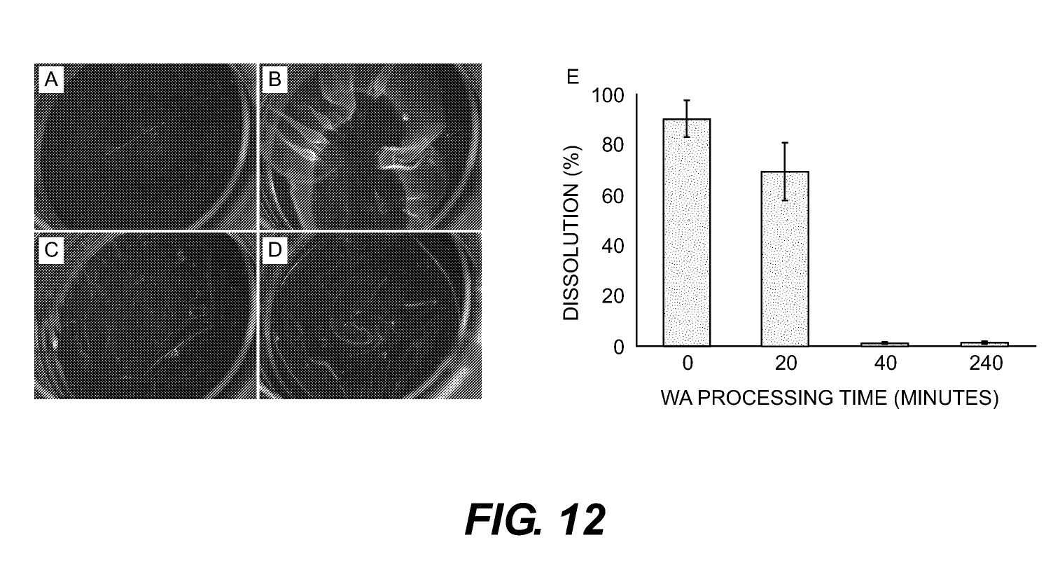

Silk films were successfully created that dissolved to varying degrees in water as a result of different WA processing time (FIGS. 12A-12D). Non-dissolved portions of the film remained translucent. Unprocessed silk films had near complete dissolution in water (FIG. 12A), while there was reduced dissolution for samples water-annealed for 20 minutes (FIG. 12B). It was shown that the edge of the silk films tended to dissolve to less of a degree than the central regions. Silk films water-annealed for 40 minutes and longer appeared to show limited dissolution in water and maintained transparency (FIGS. 12C-12D). Protein assay results conferred qualitative results indicating that protein dissolution was reduced on average with increased WA processing time (FIG. 12E).

Silk films proved to be highly transparent after WA processing as previously demonstrated. Lawrence et al., Bioactive silk protein biomaterial systems for optical devices. Biomacromolecules, 2008; 9(4):1214-20. It was found that the films could self-adhere to the cornea after both processing time points, and allowed for a non-invasive and straightforward application method of the silk film to the ocular surface. It was demonstrated that the flat silk films would readily smooth over the rabbit corneal surface as it hydrated as monitored by slit lamp photography. As the film hydrated, it began to turn into a gelatinous consistency as the material began to dissolve. This gelatinous material remained adhered to the cornea surface.

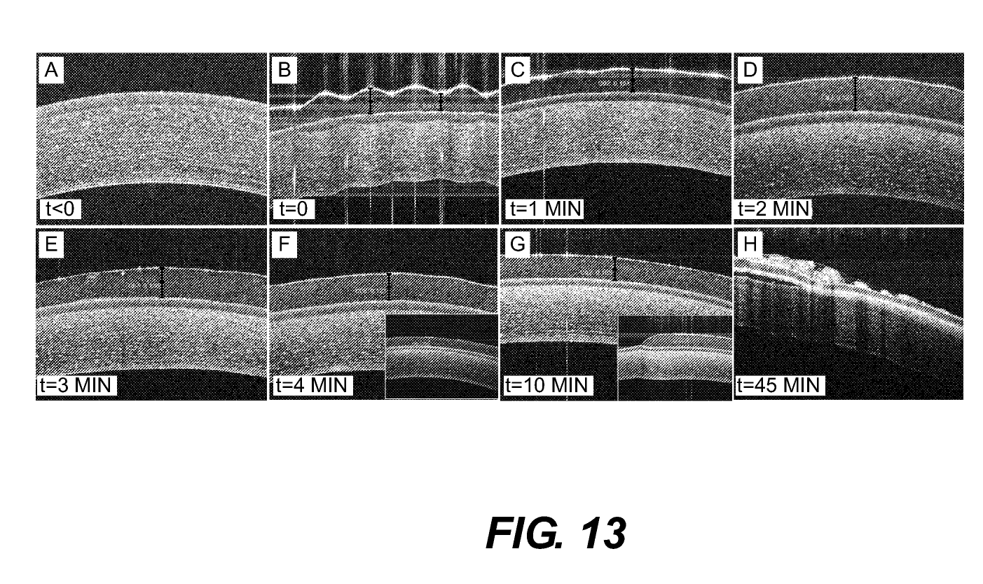

Time-lapse OCT images were taken of the untreated silk film's adhesion response to the uninjured rabbit cornea (FIG. 13A). Upon initial application the film produced standing wave morphology with peaks in thickness ranging from 79 to 114 μm in thickness (FIG. 13B). After 1-minute post-application the silk film thickness had evened out to around 100 um, which corresponded to a 25% increase in thickness (FIG. 13C). Over a 3-minute time period the silk film increased up to 136 μm in thickness, which corresponded to a 70% increase in silk film thickness post-application (FIGS. 13D-13E). The thickness of the film then began to decrease as the material began to dissolve after 4 minutes post application (FIG. 13F). After 10 minutes post-application, the silk film thickness had reduced to around 100 μm in thickness, and the edge regions appeared to maintain both consistent thickness and attachment to the corneal surface (FIG. 13G). After a total of 45 minutes upon the eye portions of the silk film remained non-dissolved and silk film particulates were spread over the cornea in various regions (FIG. 13H). The rabbit cornea appeared unaffected by the presence of the material, and the animal showed no signs of discomfort or subsequent inflammation after the silk film's application.

### Rabbit Animal Model

Three sets of animal trials were performed for silk films that were WA processed for 0, 20 and 40 minutes respectively to assess the impact of the material presence on corneal healing.

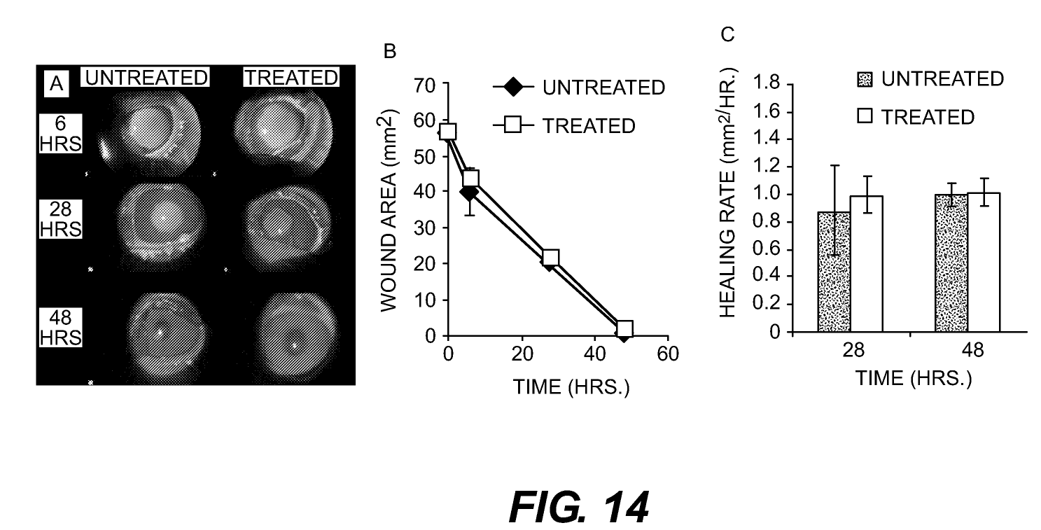

Unprocessed silk films dissolved on the ocular surface within 5 minutes post-application (FIG. 14A), and demonstrated no effect on healing when compared to the untreated control group (FIGS. 14B-14C). No silk remained after 1 hour post-application as revealed by the visual inspection of the rabbit within the cage.

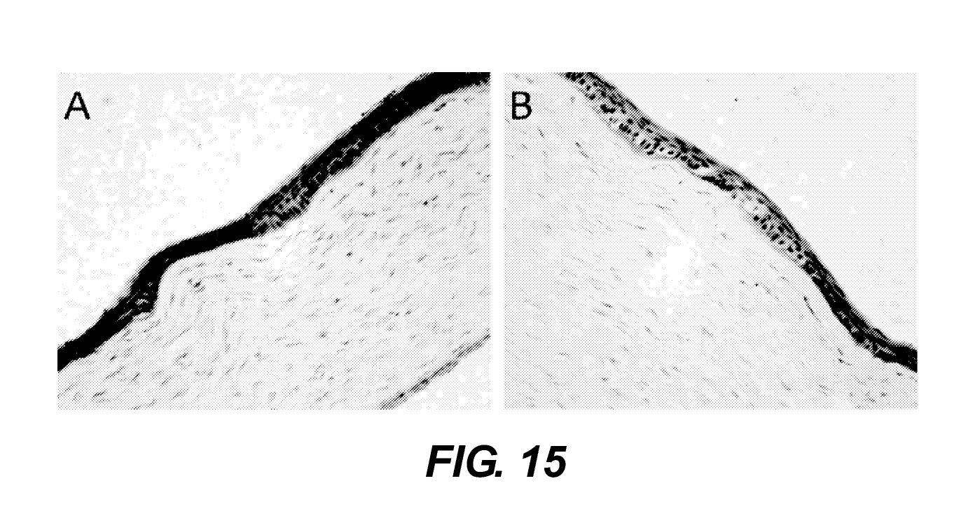

The presence of the silk film did not adversely affect the overall corneal epithelium healing 7 days post-surgery as revealed by H&E staining (FIG. 15). Corneas from both untreated and treated groups demonstrated a completely healed epithelial layer, reformation of the basement membrane, and an absence of inflammatory cells. The corneal stroma region appeared unaffected, with the absence of any neovascularization or inflammatory cells. In addition, no remnants of the silk film were observed in the histology samples.

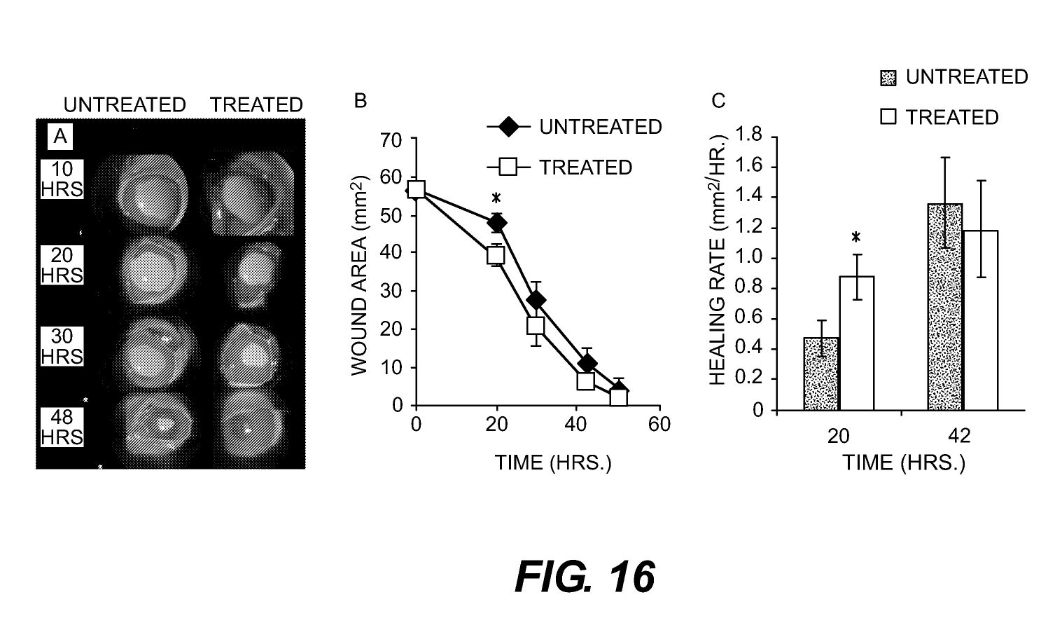

Silk films that were water annealed for 20 minutes were than applied onto the debrided epithelium (FIG. 16A). The silk film material was found to reside on the ocular surface for up to 10 hours post-procedure. In addition, the silk film treated group demonstrated a significant increase (30%, n=3, p<0.05) in healing rate over the first 24-hour period, while the film was still present on the wound bed when compared to untreated controls (FIGS. 16B-16C). After 48 hours, the silk film was no longer present on the corneal surface, and it was shown that the healing rate was similar to untreated controls.

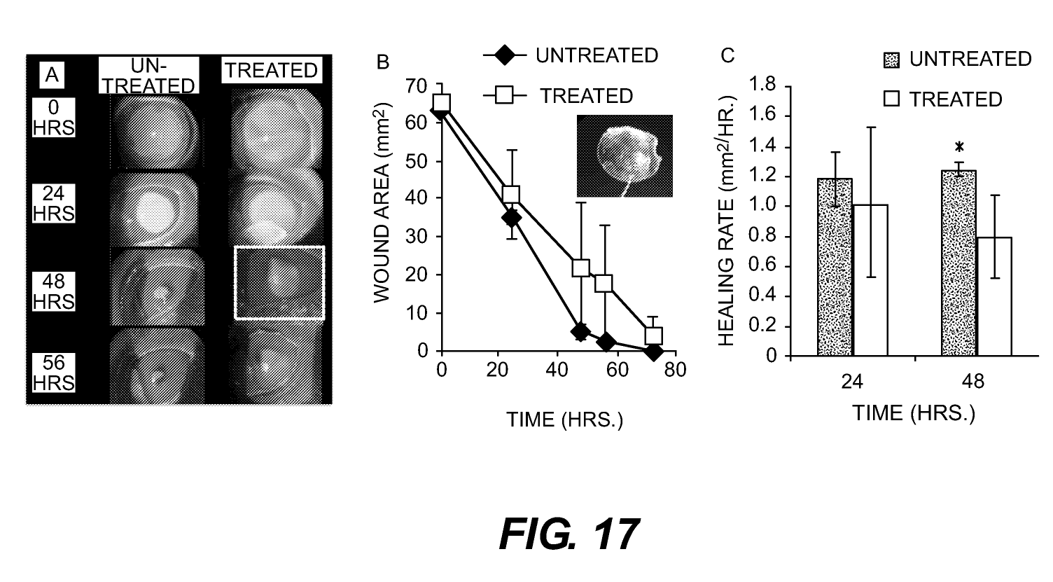

Silk films that were water annealed for 40 minutes were than applied onto the debrided epithelium (FIG. 17A). Films were still present on the corneal surface 48 hours post-procedure, at which point they were removed with forceps. Non-dissolved films were found to negatively impact healing rate when compared to untreated controls (FIG. 17B). This was also seen as a statistically significant decrease in healing rate for silk treated animals after 48 hours post-application (FIG. 17C). These results indicated that the presence of the non-dissolving silk film material was causing adverse effects on corneal healing in rabbits.

### Discussion

Silk film attachment, hydration, and dissolution was observed using OCT imaging. Results indicated that the silk film uniformly attached to the cornea surface, and produced a wave-like morphology during the initial hydration process. After 2 minutes post-application, the silk film began to hydrate and readily smoothed into an even thickness while remaining adhered to the cornea surface. This was followed by a subsequent uniform expansion of the material thickness as the silk film continued to swell from hydration. This process was rapid as the film reached its maximum thickness within a few minutes after application. The silk film appeared to both swell and simultaneously begin to dissolve, which was seen as a reduction in silk film thickness around 5 minutes after application. Film dissolution was much less rapid, and insoluble particulates remained on the eye after 45 minutes post-procedure. These results demonstrated that a self-adhering silk film could be designed to maintain residency on the cornea, while remaining attached to the epithelial surface.

Rabbit animal trials demonstrated that silk films could be used to enhance re-epithelialization after surgery by 30% if the material retained a limited period of residence time over the corneal injury site. Histology revealed that the presence of the silk protein material did not impart a negative effect on the cornea post-application. However, if the residence time of the silk film on the injury site was too extensive this appeared to decrease healing.

The scope of the present invention is not limited by what has been specifically and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

What is claimed is:

1. A degradable composition comprising fibroin, wherein fibroin comprises β-sheet conformation ranging from about 0% to about 30%, and wherein, upon contacting with a body fluid, less than about 20% (w/w) of the composition degrades after about 1 minute, and greater than about 80% (w/w) of the composition degrades after about 24 hours.

2. A degradable composition comprising fibroin, wherein the composition comprises about 1% (w/w) to about 12% (w/w) water, and wherein, upon contacting with a body fluid, less than about 20% (w/w) of the composition degrades after about 1 minute, and greater than about 80% (w/w) of the composition degrades after about 24 hours.

3. A degradable composition comprising fibroin, wherein the composition comprises about 1% (w/w) to about 12% (w/w) water, wherein fibroin comprises β-sheet conformation ranging from about 0% to about 30%, and wherein, upon contacting with a body fluid, less than about 20% (w/w) of the composition degrades after about 1 minute, and greater than about 80% (w/w) of the composition degrades after about 24 hours.

4. The composition of claim 1, wherein fibroin comprises β-sheet conformation ranging from about 5% to about 15%.

5. The composition of claim 1, wherein fibroin comprises α-helical conformation ranging from about 1% to about 80%.

6. The composition of claim 1, wherein the composition comprises about 5% (w/w) to about 10% (w/w) water.

7. The composition of claim 1, wherein upon contacting with a body fluid, less than about 20% (w/w) of the composition degrades after about 2 hours, and greater than about 80% (w/w) of the composition degrades after about 20 hours.

8. The composition of claim 1, wherein upon contacting with a body fluid, less than about 10% (w/w) of the composition degrades after about 1 minute, and greater than about 90% (w/w) of the composition degrades after about 24 hours.

9. The composition of claim 1, wherein upon contacting with a body fluid, greater than about 80% (w/w) of the composition degrades after about 10 hours.

10. The composition of claim 1, wherein upon contacting with a body fluid, greater than about 90% (w/w) of the composition degrades after about 10 hours.

11. The composition of claim 1, wherein fibroin is obtained from silkworm silk, spider silk or genetically engineered silk.

12. The composition of claim 11, wherein the silkworm silk is obtained from Bombyx mori.

13. The composition of claim 11, wherein the spider silk is obtained from Nephila clavipes.

14. The composition of claim 1, wherein fibroin is in an amount ranging from about 80% to 100%.

15. The composition of claim 1, wherein the composition is transparent.

16. The composition of claim 1, wherein the composition is opaque.

17. The composition of claim 1, wherein the composition is a film, a fiber, a foam, a hydrogel, a matrix, a mesh, a three-dimensional scaffold, a microparticle, a nanoparticle or a mat.

18. The composition of claim 17, wherein the composition is a film.

19. The composition of claim 18, wherein the film has a curved surface.

20. The composition of claim 18, wherein the film has a flat surface.

21. The composition of claim 18, wherein the film has a thickness ranging from about 1 μm to about 500 μm.

22. The composition of claim 21, wherein the film has a thickness ranging from about 10 μm to about 200 μm.

23. The composition of claim 22, wherein the film has a thickness ranging from about 50 μm to about 100 μm.

24. The composition of claim 18, wherein the film has a tensile strength ranging from about 1 to about 200 MPa.

25. The composition of claim 18, wherein the film has a tensile modulus ranging from about 0.1 to about 5 GPa.

26. The composition of claim 18, wherein the film is surface-patterned.

27. The composition of claim 18, wherein the film is smooth.

28. The composition of claim 1, further comprising a pharmacologically and/or biologically active agent.

29. The composition of claim 28, wherein the pharmacologically or biologically active agent is chosen from proteins, peptides, nucleic acids, carbohydrates, glycoproteins, lipoproteins, RNA/protein composites, cells, nucleic acid analogues, nucleotides, oligonucleotides, peptide nucleic acids, aptamers, viruses, small molecules, or combinations thereof.

30. The composition of claim 28, wherein the pharmacologically or biologically active agent is a cell.

31. The composition of claim 30, wherein the cell is chosen from epithelial cells, stem cells, smooth muscle cells, skeletal muscle cells, cardiac muscle cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid cells, parathyroid cells, adrenal cells, hypothalamic cells, pituitary cells, ovarian cells, testicular cells, salivary gland cells, adipocytes, precursor cells and mixture thereof.

32. The composition of claim 28, wherein the pharmacologically or biologically active agent is mixed with the composition.

33. The composition of claim 28, wherein the pharmacologically or biologically active agent is coated on the composition.

34. A method for treating an ocular condition in a subject comprising applying a degradable composition to an eye of the subject, wherein the composition comprises fibroin, wherein fibroin comprises β-sheet conformation ranging from about 0% to about 30%, and wherein, upon contacting with the eye, less than about 20% (w/w) of the composition degrades after about 1 minute, and greater than about 80% (w/w) of the composition degrades after about 24 hours.

35. The method of claim 34, wherein upon contacting with the eye, greater than about 80% (w/w) of the composition degrades after about 10 hours.

36. The method of claim 34, wherein the ocular condition is an ocular surface disorder.

37. The method of claim 34, wherein the ocular condition is chosen from corneal ulcer, corneal erosion, corneal abrasion, corneal degeneration, corneal perforation, corneal scarring, an epithelial defect, keratoconjunctivitis, idiopathic uveitis, corneal transplantation, dry eye syndrome, age-related macular degeneration (AMD, wet or dry), diabetic eye conditions, blepharitis, glaucoma, ocular hypertension, post-operative eye pain and inflammation, posterior segment neovascularization (PSNV), proliferative vitreoretinopathy (PVR), cytomegalovirus retinitis (CMV), endophthalmitis, choroidal neovascular membranes (CNVM), vascular occlusive diseases, allergic eye disease, tumors, retinitis pigmen-tosa, eye infections, scleritis, ptosis, miosis, eye pain, mydriasis, neuralgia, cicatrizing ocular surface diseases, ocular infections, inflammatory ocular diseases, ocular surface diseases, corneal diseases, retinal diseases, ocular manifestations of systemic diseases, hereditary eye conditions, ocular tumors, increased intraocular pressure, herpetic infections, ptyrigium (scleral tumor), wounds sustained to ocular surface, post-photorefractive keratotomy eye pain and inflammation, thermal or chemical burns to the cornea, scleral wounds, or conjunctival wounds.

38. The method of claim 34, wherein the ocular condition is caused by aging, an autoimmune condition, trauma, infection, a degenerative disorder (such as keratoconus), endothelial dystrophies, and/or a surgery.

39. The method of claim 34, wherein the composition is self-adhered or sutured to the eye.

40. The method of claim 34, wherein the silk fibroin is obtained from silkworm silk, spider silk or genetically engineered silk.

41. The method of claim 40, wherein the silkworm silk is obtained from Bombyx mori.

42. The method of claim 40, wherein the spider silk is obtained from Nephila clavipes.

43. The method of claim 34, wherein the composition is a film, a gel, a hydrogel, an ocular implant, a punctal plug, a contact lens, particles, microparticles, nanoparticles, a mucoadhesive formulation, an in-situ forming gel or film, an iontophoresis formulation, a tablet, a rod, a fiber mat, a fiber, or a patch.

44. The method of claim 43, wherein the composition is a film.

45. The method of claim 44, wherein the film has a curved surface.

46. The method of claim 44, wherein the film has a flat surface.

47. The method of claim 44, wherein the film has a thickness ranging from about 1 μm to about 500 μm.

48. The method of claim 34, wherein the composition is transparent.

49. The method of claim 34, wherein the composition is opaque.
